(19) United States
(12) Patent Application Publication
(10) Pub. No.: US 2006/0166867 A1
(43) Pub. Date: Jul. 27, 2006

(54) Novel conjugates of polysaccharides and uses thereof

(75) Inventors: Aviva Lapidot, Rehovot (IL); Ravi Hegde, Rechovot (IL); Gadi Borkow, Kfar-Gibton (IL)

Correspondence Address: Martin D. Moynihan;PRTSI, Inc., P.O. Box 16446, Arlington, VA, 22215, US

(73) Assignee: Yeda Research And Development Co. Ltd., Rehovot (IL)

(21) Appl. No.: 11/329,077

(22) Filed: Jan. 11, 2006

Related U.S. Application Data
(63) Continuation-in-part of application No. 10/831,224, filed on Apr. 26, 2004.
(60) Provisional application No. 60/465,775, filed on Apr. 28, 2003.

(51) Int. Cl. A61K 38/14; C07K 9/00

(52) U.S. Cl. 514/8; 530/322

(57) ABSTRACT

Novel conjugates composed of a saccharide-containing moiety (e.g., aminoglycosides) covalently linked to a moiety containing two or more basic amino acid residues (e.g., a polyarginine) and processes of preparing same are disclosed. Further disclosed are pharmaceutical compositions containing these conjugates and uses of these conjugates as antiviral and antibacterial agents.

This application is a continuation-in-part of U.S. patent application Ser. No. 10/831,224, filed Apr. 26, 2004, which claims the benefit of priority from U.S. Provisional Patent Application No. 60/465,775, filed Apr. 28, 2003.

### FIELD AND BACKGROUND OF THE INVENTION

The present invention relates to novel modified polysaccharides and uses thereof and, more particularly, to modified polysaccharides that can be efficiently used as anti-viral and anti-bacterial agents.

Antimicrobial agents, which are also referred to interchangeably, herein and in the art as “antibacterial agents” or “antibiotics” are an essential part of modern medicine. One of the most prevalent limitations associated with the presently available antibiotics is the evolvement of resistance thereto. Resistance factors can be encoded on plasmids or on the chromosome. Resistance may involve decreased entry of the antibiotic into the microorganism's cells, changes in the receptor (target) of the antibiotic, or metabolic inactivation thereof. Other limitations include the toxicity of antibiotics and alterations of the normal intestinal flora which may result in diarrhea or in superinfection with opportunistic pathogens. The rapid spread of antibiotic resistance in pathogenic bacteria has prompted a continuing search for new agents that exhibit antibacterial activity. Indeed, microbiologists today warn of a “medical disaster” which could lead back to the era before penicillin, when even seemingly small infections were potentially lethal. Thus, research into the design of new antibiotics is of high priority.

Aminoglycosides are known as highly potent, broad-spectrum antibiotics with many desirable properties for the treatment of life-threatening infections (Davis, B. D. Microbiol. Rev. 1987, 51, 341-350). Their history began in 1944 with streptomycin and was thereafter marked by the successive introduction of a series of milestone compounds (neomycin, kanamycin, gentamycin, tobramycin, and others), which soon established the usefulness of this class of antibiotics, particularly in the treatment of gram-negative bacillary infections (Davis (1987) supra). It is believed that aminoglycosides exert their therapeutic effect by interfering with translational fidelity during protein synthesis via interaction with the A-site rRNA on the 16S domain of the ribosome (Moazed and Noller, Nature (1987) 327, 389-394; Woodcock et al., EMBO J. (1991) 10, 3099-3103). NMR studies addressing aminoglycoside antibiotic binding to RNA suggest that rings I and II of the neomycin-class aminoglycosides are sufficient for mediating the specific interaction with the RNA (Fourmy (1998) J. Mol. Biol. 277: 347-362), whereby other rings, as well as amino groups increase RNA binding affinity (Ryu (2002) Biochemistry 41:10499-509).

However, as for most antibiotics, a major problem in the use of aminoglycosides as antibacterial agents is the development of resistance after prolonged clinical use thereof (Wright et al., Adv. Exp. Med. Biol. (1998) 456, 27-69). Presently, resistance to these agents is widespread among pathogens worldwide which severely limits their usefulness.

One way to delay the emergence of antibiotic-resistance is to develop new synthetic materials that can selectively inhibit bacterial enzymes, via novel mechanisms of action. This approach is both time-consuming and financially prohibitive, and yet for the time being it remains indispensable. Another less costly and less time-consuming approach is to restore the usefulness to antibacterial agents that have become compromised by resistance, by introducing certain modifications to their structures. The remarkable advances in recent years in elucidating the mechanisms of resistance to various clinical antibiotics in the molecular level provide complementary tools to this approach via structure-based and mechanism-based design.

Another important disease which may be treated with aminoglycosides is acquired immunodeficiency syndrome (AIDS). It is a fatal human disease, which has affected numerous individuals worldwide. The causative agent of AIDS is the Human Immunodeficiency Virus (HIV). One approach for AIDS drug therapy is to target viral proteins in an attempt to inhibit or halt viral replication. In the replication stage of HIV-1, two pairs of proteins and the corresponding RNAs play a critical role. One of these is a trans-activator protein (TAT) and its responsive mRNA fragment, trans-activator-responsive element (TAR), and the other is a retro viral protein (REV) and its responsive mRNA, REV responsive element (RRE) (Cullen and Green, Cell (1989) 58, 423; Sharp and Marciniak, Cell (1989) 59, 229; Malim and Cullen, Cell (1991) 65, 241). Different studies have shown that several aminoglycosides are known to bind either TAR RNA or RRE RNA and disturb the RNA-protein binding (Zapp et al., Cell (1993) 74, 969; Wang et al., Biochemistry (1998) 37, 5549).

As in the field of antibiotics, there is a continuing struggle to overcome the emergence of viral drug-resistant strains. Current strategies for coping with the developed resistance to antiviral agents include combination drug therapies, namely drugs aimed against different viral proteins or drugs aimed at more than one site on the same protein. However, although this approach has been successful in delaying disease progression and improving the quality of life of AIDS patients, significant problems still remain, including drug toxicity and emergence of additional resistant viral strains (see, for example, Birch (1998) AIDS 12:680-681; Roberts (1998) AIDS 12:453-460).

To tackle the problem of antibiotic and antiviral resistance in natural aminoglycosides, many structural analogs of aminoglycosides have been synthesized over the past decade (for a recent review see: Ye, X.-S.; Zhang, L.-H. Curr. Med. Chem. (2002), 9, 929-939). In the majority of these studies a minimal structural motif, which is common for a series of structurally related aminoglycosides, has been identified and used as a scaffold for the construction of diverse analogs as potential new antibiotics. Some of the designed structures show considerable antibacterial activities.

Although it has been established that aminoglycosides, as well as structurally modified derivatives thereof, serve as important antibacterial and antiviral agents, aminoglycosides are hardly lipid soluble and are therefore unable to pass through the cell membranes and reach the target site. The impermeability of the cell membrane to aminoglycosides then results in an increased resistance to aminoglycosides. Thus it is advisable to develop ways to overcome this impermeability.

One way of overcoming this problem, and hence an important feature in the development of new drugs, is using the capability of many peptides, many of which are present in viral proteins, to cross the biological membranes of a variety of cell types.

Arginine- and lysine-rich basic peptides include a common motif of RNA recognition by proteins. Thus, for example, HIV TAT and REV proteins mediate their interactions with the viral RNAs via arginine rich motif (Weeks, Science (1999) 249:1281-1285). Although the dominant contributions of the arginine side-chains may differ between complexes, the ability of the guanidine groups of the arginine side chains to be involved in the electrostatic interactions, hydrogen bond formation, π-π and stacking interactions make arginine an important moiety for RNA recognition (Cheng, Curr. Opin. Struct. Biol. (2001) 11:478-484). Arginine-rich RNA-binding peptides and peptidomimetics have provided a good scaffold for RNA-targeted drug design since they are short, conformationally diverse and contact RNA with high affinity and specificity (see Borkow and Lapidot, Current Drug Targets—Infectious Disorders (2005) Vol. 5, p. 3-15; Litovchick, (1999) FEBS Lett. 445:73-79; Lapidot, A.; Litovchick, A. Drug Development Research (2000), 50, 502; Litovchick, A.; Lapidot, A.; Eisenstein, M.; Kalinkovich, A.; Borkow, G. Biochemistry (2001) 40 (51), 15612-15623; Lapidot, A.; Vijayabaskar, V.; Litovchick, A.; Yu, J.; James, T. L. FEBS Lett. (2004) 577, 414; Litovchick, A.; Evdokimov, A. G.; Lapidot, A. Biochemistry (2000) 39(11), 2838). For example, the HIV-1 TAT protein which is essential for HIV-1 replication is also capable of translocating through host cell membrane. TAT residue 48-60 which consists of eight positively charged amino acids, six arginine and two lysine residues, rapidly translocates through the plasma membrane and accumulates in the cell nucleus (Fawell et al., J. Proc. Natl. Acad. Sci. U S. A. (1994) 99, 664; Vives et al., J. Biol. Chem. (1997) 272, 16010; Nagahara et al. Nat. Med. (1998) 4, 1449; Schwarze et al., Science, (1999) 285, 1569). This finding led to the assumption that charged residues in membrane translocational regions have a critical role in membrane penetration (O'Brien et al., J. Virol. (1996) 70, 2825).

A co-inventor of the present invention, Prof. Lapidot, and co-workers have previously suggested that the RNA binding ability of polysaccharides in general and aminoglycosides in particular can be combined with the specific binding of arginine moiety to HIV-1 TAR RNA, and have thus prepared aminoglycoside-arginine and acetamidine conjugates (AACs) by substituting the free amino groups on the aminoglycoside by arginine or acetamidine groups (see, for example, WO 00/39139; U.S. Pat. No. 6,642,365; EP Patent Application No. 1140958 (recently granted); Litovchick et at. (1999) supra; Lapidot (2000) supra; and Litovchick et al. (2000) supra).

The conjugates described in, for example, U.S. Pat. No. 6,642,365 have been collectively represented by the following general Formula:

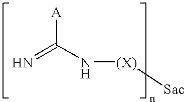

wherein A is CH₃ or NH₂; X is a linear or branched C₁-C₈ alkyl chain; n is an integer equal to or greater than 1; and Sac is the residue of a mono- or oligo-saccharide.

Some exemplary AACs are: NeoR1, a 1:1 mixture of two mono-arginine neomycin conjugates; ParomR1, a mono-arginine paromomycin; NeamR1, a mono-arginine neamine conjugate; NeoR2, a di-arginine neomycin conjugate; R3G, a tri-arginine gentamycin C1 conjugate; NeamR4, a tetra-arginine neamine conjugate; R4K, a tetra-arginine kanamycin A; ParamoR5, a penta-arginine paromomycin, NeoR6, a hexa-arginine neomycin B, and their mono-arginine conjugates (Lapidot (2002) supra, Litovchick (1999) supra; Litovchick, A.; Lapidot et al. (2001) supra; Dereu, N. J. Med. Chem. (1996) 39(5), 1069; U.S. Pat. No. 6,642,365).

The chemical structure of an exemplary AAC, a hexaarginine neomycin B conjugate (NeoR6), which was found highly potent as an anti-viral agent, is presented below:

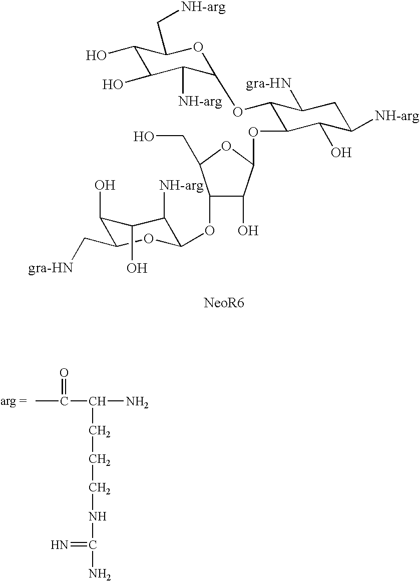

These AACs were designed to bind HIV TAR RNA and to inhibit trans-activation by TAT protein. These AACs were found to act as antagonists of the HIV-1 TAT protein basic domain and structurally are peptidomimetic compounds with different aminoglycoside cores and different numbers of arginines (Litovchick (1999), supra; Litovchick et al. (2000) supra; Lapidot (2000) supra; Litovchick et al. (2001) supra). Along with inhibition of TAT trans-activation step in HIV life cycle, AACs exert a number of other activities, closely related to TAT antagonism. For example, hexa-arginine neomycin B conjugate (NeoR6) inhibits the several functions of extra cellular TAT protein including upregulation of the HIV-1 viral entry co-receptor (CXCR4), increase of viral production, suppression of CD3-induced proliferation of lymphocytes, and upregulation of CD8 receptor (Litovchick (2001) supra). It was recently shown that NeoR6 and a tri-arginine-gentamycin conjugate (R3G) inhibit binding of HIV particles to cells, presumably by blocking the CXCR4 co-receptor (Litovchick (2000) supra; Litovchick (2001) supra). This was further substantiated by the finding that NeoR6 competes with the binding of the monoclonal antibody 12G5 to CXCR4, and CXCR4-SDF-1α binding (Litovchick (2001) supra) and inhibits elevation of intracellular Ca²⁺ induced by SDF-1α (Cabrera (2002) Antiviral Res. 53:1-8; Cabrera (2000) AIDS Res. Hum. Retroviruses 16:627-634; and also reviewed in Borkow, G.; Lara, H. H.; Lapidot, A. Biochem. Biophys. Res. Commun. 2003, 312(4), 1047). Several studies have demonstrated that both the aminoglycoside core and the number of arginines attached to the specific aminoglycoside, plays an important role in the antiviral potency of the AACs (Borkow, G.; Vijayabaskar, V.; Lara, H. H.; Kalinkovich, A.; Lapidot, A. Antiviral Res. (2003a) 60(3), 181; Lapidot et al. (2004) supra). Thus, for example, NeoR6, the hexa-arginine-neomycin B conjugate, was found to have a higher antiviral activity, as compared to the tri-arginine-gentamycin R3G, against wild-type and NeoR6 resistant isolates (an EC50 of 1.9 and 4.1 μM, respectively). Interestingly, it has been found that although both R3G and NeoR6 interact with CXCR4 (Lapidot (2001) supra; Borkow et al. (2003a) supra) and with HIV-1 TAR RNA (Lapidot (2001) supra, Borkow, G.; Lara, H. H.; Lapidot, A. Biochem. Biophys. Res. Commun. 2003, 312(4), 1047; Borkow et al. (2003a) supra), no mutations in gp102 or in gp41 are found in R3G resistant isolates (R3G^{res}) (Borkow and Lapidot, unpublished data), as were found for NeoR6^{res} isolates (Lapidot (2000) supra; Hotzel, I. AIDS Res. Hum. Retroviruses (2003) 19(10), 923). Furthermore, while the NeoR6^{res} isolates were approximately 50 times more resistant than the wild-type virus to NeoR6, they were only about 5 times more resistant than the wild-type virus to R3G. In contrast, R3G^{res} isolates were almost as sensitive as the wild-type virus to NeoR6 (Borkow and Lapidot, unpublished data). Taken together, these data support the notion that different AACs exert antiviral activity via different mechanisms, at least during the viral entry step.

Noteworthy is that AACs penetrate a variety of mammalian cells, including neurons and accumulate intracellularly (Litovchick (2001) supra; Litovchick (1999) supra; and Cabrera (2000) supra). In particular, NeoR6 was shown to cross the blood brain barrier when administered systematically and to thereby penetrate various brain tissues (Catani, M. V.; Corasaniti, M. T.; Ranalli, M.; Amantea, D.; Litovchick, A.; Lapidot, A.; Melino, G. J. Neurochem. (2003) 84(6), 1237; Borkow et al. (2003a) supra; and Borkow, G.; Lapidot, A. Curr. Drug Targets—Infectious Disorder (2005) 5, 3).

These features render AACs multifunctional HIV-1 antagonists and therefore a highly important novel class of anti viral drugs.

Additional studies have also demonstrated that AACs (such as, for example, NeoR6 and the tri-arginine gentamycin conjugate (R3G)) are able to elicit inhibition of bacterial RNAse P, and to a lesser extent, of mammalian RNAse P (see, for example, WO 03/059246). The inhibitory activity of these conjugates was found far more significant than that elicited by their unconjugated aminoglycoside counterparts.

In view of the ever-expanding roles of AACs in antibacterial and antiviral therapies, it is highly desirable to further elucidate the structural functional relationship of AAC binding to RNA, as well as the mechanism of inhibiting HIV-1 cell entry, in order to design and identify antiviral and antibacterial drugs with improved therapeutic efficacy and reduced cytotoxicity.

While conceiving the present invention, it was hypothesized that conjugates of polysaccharides in general and aminoglycosides in particular and a moiety that contains a plurality of arginine residues attached to one or more of the saccharide, would exert the desired improved performance.

The presently utilized AACs include a single arginine residue that is attached to one or more saccharide units in the polysaccharide.

The underlying basis of this hypothesis was derived from the recent findings regarding the use of oligoarginines for drug transportation across the cell membrane, blood brain barrier and as a delivery vector for genes, proteins, peptides, particles etc. has been reported (see, for example, Tung and Weissleder, Adv. Drug Delv. Rev., (2003), 55, 281). Thus, for example, it was reported that oligomers of arginine composed of six or more amino acids, alone or covalently attached to a variety of small molecules, efficiently cross the cell membrane (D M. H. Nelson et al. (2005) Bioconjugate Chem, 16, 959-966). It has further lately been found that the free 9-arginine-polymer serves as an anti-HIV-1 drug (W. A. O'Brien et al. (1996) supra). Moreover, positively charged amino acid peptides, in particular polyarginines such as ALX40-4C (Na-acetyl-nona-D-arginine amide), initially designed as an inhibitor of HIV-TAT binding to the viral RNA transactivator responsive element (TAR), was identified as an inhibitor of the co-receptor CXCR4 and was found to inhibit infection exclusively by blocking virus-CXCR4 interaction (Doranz, et al. J. Expt. Med. (1997) 186, 1395; Doranz et al., AIDS Res. Hum. Retroviruses. (2001), 17, 475).

### SUMMARY OF THE INVENTION

While reducing the present invention to practice, the present inventors have designed and successfully prepared a series of novel conjugates of aminoglycosides, each having a plurality of arginine residues attached to a single saccharide unit of the aminoglycoside. These novel conjugates, which are referred to herein as pAACs, were found highly potent antiviral and antibacterial agents. These pAAC conjugates exemplify the potential of conjugates of poly- and oligosaccharides and a moiety that contains a plurality of basic amino acids to serve as a novel class of antiviral and antibacterial agents.

Thus, according to one aspect of the present invention there is provided a conjugate comprising a first moiety and a second moiety being covalently linked therebetween, wherein said first moiety includes at least one saccharide unit and said second moiety includes two or more basic amino acid residues.

According to further features in preferred embodiments of the invention described below, the first moiety is selected from the group consisting of a monosaccharide, an oligosaccharide and a polysaccharide. Preferably, the oligosaccharide is an aminoglycoside antibiotic. More preferably, the aminoglycoside antibiotic is selected from the group consisting of neomycin, kanamycin, sisomycin, fortimycin, paromomycin, neamine and gentamycin.

According to still further features in the described preferred embodiments the second moiety is linked to an aminoalkyl group of the aminoglycoside. Preferably, the second moiety is linked to the aminoalkyl group via an amide bond.

According to still further features in the described preferred embodiments the second moiety comprises six or more basic amino acid residues.

According to still further features in the described preferred embodiments the second moiety comprises from 6 to 9 basic amino acid residues.

Hence, according to yet another aspect of the present invention there is provided a conjugate comprising a first moiety and a second moiety being covalently linked therebetween, wherein the first moiety includes at least one saccharide unit and the second moiety includes from 6 to 9 basic amino acid residues.

According to further features in preferred embodiments of the invention described below, the second moiety comprises 6 basic amino acid residues or 9 basic amino acid residues.

According to still further features in preferred embodiments of the invention described below, the second moiety is a peptide which comprises the two or more basic amino acid residues.

According to still further features in the described preferred embodiments the second moiety is a peptide which comprises six or more basic amino acid residues.

According to still further features in the described preferred embodiments the second moiety is a peptide which comprises from 6 to 9 basic amino acid residues.

Preferably, the peptide consists essentially of the basic amino acid residues.

According to still further features in the described preferred embodiments the basic amino acid residues are selected from the group consisting of arginines, lysines, histidines, omithines and any combinations thereof.

According to still further features in the described preferred embodiments the basic amino acid residues are arginine residues.

According to still further features in the described preferred embodiments the basic amino acid residues are selected from the group consisting of L-amino acid residues, D-amino acid residues and combinations thereof.

According to further features in preferred embodiments of the invention described below, the basic amino acid residues are D-amino acid residues.

According to still further features in the described preferred embodiments the basic amino acid residues are L-amino acid residues.

According to further features in preferred embodiments of the invention described below, the conjugate further comprises at least one organic residue having a molecular weight of about 55 daltons.

According to yet another aspect of the present invention there is provided a process of preparing the conjugates described hereinabove, the process comprising: coupling a first compound having at least one saccharide unit and a second compound having two or more basic amino acid residues, thereby obtaining the conjugate.

Preferably, the coupling is effected in the presence of a coupling agent. More preferably, the coupling agent is a peptide coupling agent.

According to further features in preferred embodiments of the invention described below, the at least one saccharide unit comprises an aminoalkyl group and the coupling is effected via the aminoalkyl group, such that the process further comprises, prior to the coupling: providing a compound having at least one saccharide unit and at least one aminoalkyl group attached to the saccharide unit, wherein any non-alkylamino groups in the compound are protected.

Preferably, providing the compound having at least one saccharide unit and at least one aminoalkyl group attached to the saccharide unit, wherein any non-alkylamino groups in the compound are protected comprises: selectively protecting the at least one aminoalkyl group with a first protecting group; selectively protecting the non-alkylamino groups with a second protecting group; and selectively removing the first protecting group.

Preferably, the first protecting group is derived from a bulky protecting compound. More preferably, the bulky protecting compound is selected from the group consisting of tritylhalide and N-(tert-butoxycarbonyloxy)-5-norbornene-endo-2,3-dicarboximide.

According to still further features in preferred embodiments of the present invention described below, the compound having the basic amino acids comprises at least one third protecting group protecting at least one functional group in the compound, and the process described hereinabove further comprises removing the third protecting group. Preferably, removing the third protecting group is effected subsequent to the coupling.

According to yet another aspect of the present invention there is provided a pharmaceutical composition comprising, as an active ingredient, any of the conjugates described hereinabove and pharmaceutically acceptable carrier.

According to further features in preferred embodiments of the invention described below, the pharmaceutical composition is packaged in a packaging material and identified in print, in or on the packaging material, for use in the treatment of a medical condition associated with an infectious microorganism. Preferably, the infectious microorganism is selected from the group consisting of a virus and a bacterial strain, as detailed hereinbelow.

According to still further features in the described preferred embodiments the pharmaceutical composition further comprises at least one antiviral agent.

According to still further features in the described preferred embodiments the pharmaceutical composition further comprises at least one antibacterial agent.

According to still another aspect of the present invention there is provided a method of treating a medical condition associated with an infectious microorganism, the method comprising administering to a subject in need thereof a therapeutically effective amount of any of the conjugates described hereinabove.

According to still further features in the described preferred embodiments the method further comprises administering to the subject at least one antiviral agent.

According to still further features in the described preferred embodiments the method further comprises administering to the subject at least one antibacterial agent.

According to an additional aspect of the present invention there is provided a use of the conjugates described hereinabove in the treatment of a medical condition associated with an infectious microorganism.

According to yet an additional aspect of the present invention there is provided a use of the conjugates described hereinabove in the preparation of a medicament.

According to still an additional aspect of the present invention there is provided a use of the conjugates described hereinabove in the preparation of a medicament for the treatment of a medical condition associated with an infectious microorganism.

According to further features in preferred embodiments of the invention described below, the infectious microorganism is selected from the group consisting of a virus and a bacterial strain.

In one embodiment, the virus is HIV and the medical condition is, for example, AIDS and an AIDS manifestation.

In another embodiment, the bacterial strain is a resistant bacterial strain such as, for example, a Gram positive strain and a Gram negative strain. Examples of gram negative bacterial strains include, without limitation, Escherichia coli, Pseudomonas aeruginosa, Klebsiella pneumoniae, Proteus mirabilis, Acinetobacter baumannii, Moraxella catarrhalis, Serratia marcescens, Enterobacter cloacae and Enterobacter aerogenes. Examples of gram positive bacterial strains include, without limitation, Staphylococcus aureus, Staphylococcus epidermidis, Streptococcus pyogenes, Streptococcus bovis, Streptococcus Pneumoniae, Streptococcus Pyogenes, Streptococcus Agalactiae, Bacillus subtilis, Enterococcus faecalis, Enterococcus faecium and Listeria monocytogenes.

The present invention successfully addresses the shortcomings of the presently known configurations by providing novel conjugates of a plurality of basic amino acid residues (e.g., a polyarginine) and a saccharide (e.g., an aminoglycoside), which are characterized by high inhibitory activity of viral and bacterial infectivity, as well as by high cellular intake, and are therefore far superior to the presently known anti-viral and anti-bacterial agents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

As used herein, the term “comprising” means that other steps and ingredients that do not affect the final result can be added. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

The term “method” or “process” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the singular form “a,” “an,” and “the” include plural references unless the context clearly dictates otherwise. For example, the term “a compound” or “at least one compound” may include a plurality of compounds, including mixtures thereof.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a schematic illustration depicting a stepwise process of site-specific conjugation of polyarginines to aminoglycosides, so as to produce preferred polyarginine-aminoglycoside conjugates according the present embodiments;

FIGS. 2A-F present confocal microscopy images of cMAGI cells, following a 30 minutes incubation with 15 μM and 5 μM of the L-Arg-9-mer (Compound 5, FIG. 2A at 15 μM, FIG. 2D at 5 μM), P55 (FIG. 2B at 15 μM, FIG. 2E at 5 μM) and L-Arg-9-mer-neomycin (Compound 8, FIG. 2C at 15 μM, FIG. 2F at 15 μM, FIG. 2D at 5 μM at 5 μM), following a 30 minutes incubation; and

FIGS. 3A-F present flow cytometry graphs depicting the competitive binding to CXCR4 on MT2 cells of 12G5 mAb and exemplary polyarginines and pAACs according to the present embodiments: D-Arg-6-mer (Compound 9, FIG. 3A), 6-D-Arg-neamine (Compound 10, FIG. 3B), 6-D-Arg-neomycin (Compound 11, FIG. 3C), D-Arg-9-mer (Compound 11, FIG. 3D), 9-D-Arg-neamine (Compound 12, FIG. 3E), and 9-D-Arg-neomycin (Compound 13, FIG. 3F).

### DESCRIPTION OF THE PPEFERRED EMBODIMENTS

The present invention is of novel modified polysaccharides which can be used as antiviral and anti-bacterial agents. Specifically, the present invention is of (i) novel conjugates composed of a saccharide-containing moiety and a moiety that includes a plurality (two or more) basic amino acids; (ii) a novel synthesis methodology for generating these conjugates; (iii) pharmaceutical compositions containing these conjugates; and (iv) uses of these conjugates for treating bacterial infections and viral infections such as AIDS.

The principles and operation of the compounds, processes, compositions and uses of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As discussed hereinabove, aminoglycosides are known as highly potent, broad-spectrum antibiotics in the treatment of life-threatening infections (Davis, 1987, supra). However, as for most antibiotics, a major problem in the use of aminoglycosides as antibacterial agents is the development of resistance after prolonged clinical use thereof (Wright, 1998, supra).

Another important disease which may be treated with aminoglycosides is acquired immunodeficiency syndrome (AIDS), for which HIV is the causative agent. Vast amounts of financial and human resources are currently invested into finding new therapies and new drugs, which may provide some assistance in combating the HIV virus.

In order to overcome the limitations associated with the presently used therapies, many structural analogs of aminoglycosides have been synthesized over the past decade (see, for example, Ye and Zhang, 2002, supra). These analogs, however, are typically characterized, inter alia, by insufficient activity and/or low cell permeability.

Recently, the trans activating region (TAR) RNA and the REV responsive element (RRE), both responsible for gene regulation in HIV, have been identified as possible RNA-based drug targets. Interestingly, arginine- and lysine- rich basic peptides include a common motif of RNA recognition by proteins. Thus, for example, it was suggested that HIV TAT and REV proteins mediate their interactions with the viral RNAs via arginine rich motif (Weeks, 1999, supra). It was further assumed that charged residues in membrane translocational regions have a critical role in membrane penetration (O'Brien et al., 1996, supra), and that therefore basic amino acids, such as arginine, which are positively charged under cell conditions, may have an advantage in being able to cross membrane barriers.

The present inventors have previously suggested that the RNA binding ability of aminoglycosides can be combined with the specific binding of arginine moiety to HIV-1 TAR RNA. WO 00/39139, U.S. Pat. No. 6,642,365 and EP Patent Application No. 1140958, for example, which are incorporated by reference as if fully set forth herein, teaches arginine- and acetamidino-aminoglycoside conjugates (AACs), which were prepared by substituting all the free amino groups on the aminoglycoside by arginine groups, up to six such substitutions within the saccharide. These conjugates were found highly active in inhibiting HIV infectivity.

WO 03/059246, which is also incorporated by reference as if fully set forth herein, further teaches the use of the conjugates taught in WO 00/39139, U.S. Pat. No. 6,642,365 and EP Patent Application No. 1140958 as antibacterial agents, and particularly as efficient agents in treating bacterial infections caused by resistant strains.

In each of these applications, there are taught conjugates in which a single residue of the arginine or the acetamidine is attached to one or more positions of the saccharide.

As detailed in the Background section above, positively charged peptides were found to be effective viral inhibitors (Doranz, et al., (1997) (2001), supra). Furthermore, positively charged peptides have been reported to effectively cross cell membranes (Tung and Weissleder, (2003) supra). Thus, it has been suggested that charged peptides can serve as a delivery vector for genes, proteins, peptides, particles etc.

U.S. patent application Ser. No. 10/831224 (having the Publication No. 2004/0229265), of which the instant application is a continuation-in-part, teaches a selective synthesis methodology, which enables the preparation of AACs that bear a predetermined number of arginine residues attached thereto. Thus, for example, using this methodology, aminoglycosides conjugated at a pre-selected position to a single arginine residue (e.g., NeoR1, ParamR1 and NeamR1) have been prepared. While comparing the antiviral activity of these conjugates to the known NeoR6 conjugate, the superior activity of the latter has been demonstrated.

In a search for novel compounds with improved antiviral and antibacterial performance, the present inventors have envisioned that conjugation of saccharides, such as aminoglycosides, with a polymeric chain that contains a plurality of basic amino acid residues such as arginine residues, would combine the beneficial therapeutic effect of the presently known AACs and the therapeutic and penetrative effect of a polymeric basic amino acids chain and thus would result in highly potent therapeutic agents.

Since the HIV TAT and REV molecules are polypeptides, and further since, as previously taught in, for example, WO 00/39139 and U.S. Pat. No. 6,642,365, the amino acid moiety of aminoglycoside conjugates is designed to mimic TAT and REV binding to viral RNA, inclusion of basic amino acids is thought to increase the affinity of such aminoglycoside conjugates to the viral target. Furthermore, at cell conditions, the basic amino acids are positively charged, and this is thought to enable such aminoglycoside conjugates to better cross the cell membrane, and reach the target bacterial or viral cells.

Thus, it was envisioned that attaching a polymeric chain which comprises a plurality of basic amino acid residues (e.g., a peptide of two or more basic amino acid residues) to a saccharide unit (either per se or as a part of an oligo- or polysaccharide) would result in improved efficacy of the resulting conjugate as compared with the previously known AACs, as well as with other presently known anti-viral and anti-bacterial agents.

To this end, the present inventors have designed a novel methodology for the site-specific preparation of conjugates of arninoglycosides and polyarginines (containing two or more arginine residues). While reducing the present invention to practice, exemplary conjugates of aminoglycosides and polyarginines (e.g., di-arginine, hexa-arginine or nona-arginine peptides) have been successfully prepared using this methodology. These conjugates are referred to herein interchangeably as polyarginine conjugated aminoglycosides, polyarginine aminoglycoside conjugates or are abbreviated as pAACs).

The structures of exemplary polyarginine conjugated aminoglycosides (pAACs), prepared and practiced by the present inventors, are collectively presented in Scheme I below.

SCHEME I

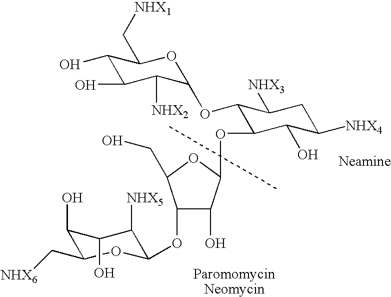

| Compound (No.) | X1 | X2 | X3 | X4 | X5 | X6 |
|---|---|---|---|---|---|---|
| Neamine | H | H | H | H | — | — |
| L-Arg-2-mer-neamine (21) | R2-NH2 | H | H | H | — | — |
| L-Arg-6-mer-neamine (2) | R6-NH2 | H | H | H | — | — |
| L-Arg-9-mer-neamine (6) | R9-NH2 | H | H | H | — | — |
| D-Arg-6-mer-neamine (10) | r6-NHAc | H | H | H | — | — |
| D-Arg-9-mer-neamine (13) | r9-NHAc | H | H | H | — | — |
| Neomycin | H | H | H | H | H | H |
| L-Arg-6-mer-neomycin (4) | R6-NH2 | H | H | H | H | H |
| L-Arg-9-mer-neomycin (8) | R9-NH2 | H | H | H | H | H |
| D-Arg-6-mer-neomycin (11) | r6-NHAc | H | H | H | H | H |
| D-Arg-9-mer-neomycin (14) | r9-NHAc | H | H | H | H | H |
| Paromomycin | OH | H | H | H | H | H |
| L-Arg-6-mer-paromomycin | OH | H | H | H | H | R6-NH2 |
| (3) | | | | | | |
| L-Arg-9-mer-paromomycin | OH | H | H | H | H | R9-NH2 |
| (7) | | | | | | |
| NeoR6 | R | R | R | R | R | R |
| (see, U.S. Pat. No. 6,642,365) | | | | | | |

R=L-arginine residue; r=D-arginine residue

As shown in Scheme 1 above, while designing and practicing the novel aminoglycoside polyarginine conjugates, the present inventors have focused on such conjugates which comprise 6 or 9 arginine residues.

The present inventors have previously shown that NeoR6 (presented above) was the most efficient anti-HIV-1 compound among all the previously synthesized AACs. It was therefore envisioned that conjugates which include a polymeric chain that comprises 6 arginine residues (as in NeoR6) would be highly efficient agents.

It was further shown that positively charged amino acid peptides, in particular poly-arginines, are inhibitors of the coreceptor CXCR4 and thus selectively affect an infection by selectively blocking virus-CXCR4 interactions. Specifically, the free 9-arginine-polymer was found to serve as an anti-HIV-1 drug. Thus, it was further envisioned that such conjugates, which include a polymeric chain that comprises 9 arginine residues (as in the above described anti-HIV agent) would also be highly efficient agents.

As is further demonstrated in the Examples section that follows, these exemplary novel conjugates were indeed found to act as efficient antiviral and antibacterial agents, having a significant cellular uptake and high activity against resistant infectious species, demonstrating the potential efficacy of the novel family of conjugates presented herein.

Thus, according to one aspect of the present invention there is provided a conjugate comprising a first moiety and a second moiety being covalently linked therebetween. The first moiety includes at least one saccharide unit and the second moiety includes two or more basic amino acid residues.

As used herein the term “moiety” describes a residue that is derived from a biologically active compound, which retains its activity. As is well accepted in the art, the term “residue” refers herein to a major portion of a molecule which is covalently linked to another molecule.

The first moiety, containing one or more saccharide unit(s), is also referred to herein interchangeable as a saccharide-containing moiety.

In each of the conjugates presented herein, the saccharide unit may form a part of a monosaccharide residue, an oligosaccharide residue or a polysaccharide residue.

As is known in the art, monosaccharides consist of a single saccharide molecule which cannot be further decomposed by hydrolysis. Representative examples of monosaccharides include, without limitation, pentoses such as, but limited to, arabinose, xylose, and ribose.

Oligosaccharides are commonly defined in the art and herein as being composed of up to nine saccharide units (see, for example, Roberts, J. D., and Caserio, M. C., Basic Principles of Organic Chemistry (1964) p. 615). Representative examples include, without limitation, disaccharides such as, but not limited to, sucrose, maltose, lactose, and cellobiose; trisaccharides such as, but not limited to, mannotriose, raffinose and melezitose; and tetrasaccharides, such amylopectin, Syalyl Lewis X (SiaLex) and the like.

The term “polysaccharide” as used herein is meant to include compounds composed of 10 saccharide units and up of hundreds and even thousands of monosaccharide units per molecule, which are held together by glycoside bonds and range in their molecular weights from around 5,000 and up to millions of Daltons. Examples of common polysaccharides include, but are not limited to starch, glycogen, cellulose, gum arabic, agar and chitin.

Alternatively, the saccharide can be a saccharine derivative such as, but not limited to, glucosides, ethers, esters, acids and amino saccharides.

According to preferred embodiments of the present invention, the second moiety is linked to a single saccharide unit of the first moiety.

Preferably, the second moiety is linked to a pre-selected position of the saccharide-containing moiety. Thus, for example, in cases where the first moiety is an oligosaccharide or a polysaccharide, the second moiety is linked to a single, pre-selected saccharide unit thereof, preferably at a pre-selected position of the pre-selected saccharide unit.

As is described in detail hereinbelow, such a selective attachment of the second moiety to the saccharide-containing moiety can be readily effected in cases where an aminoalkyl group is present within a saccharide unit in the saccharide-containing moiety.

Thus, according to preferred embodiments of this aspect of the present invention, the second moiety is linked to an aminoalkyl group present within the saccharide unit. The selective attachment can be effected while utilizing the superior reactivity of such an aminoalkyl group, as compared to other amine or other functional groups of the saccharide-containing moiety.

Conjugates in which the second moiety is attached to an aminoalkyl group of the first moiety are also referred to herein, interchangeable, as “amino-modified oligo- or poly-saccharides”.

Thus, a saccharide unit in such a conjugate is modified at its alkylamine group by being attached to the second moiety.

The conjugation of the second moiety to such an aminoalkyl group is further advantageous since it allows a more flexible interaction of the second moiety (e.g., a positively charged peptide) with the target (e.g., a specific site of the coreceptor CXCR4 or HIV Tar and/or RRE) thereof.

The second moiety is linked to the first moiety via a covalent bond formed between functional groups that are present within the moieties. Thus, the bond can be, for example, an amide (formed between an amine and carboxy), an imine (formed between amine and aldehyde), an ester (formed between hydroxy and carboxy), a thioester (formed between a thiol and carboxy), and the like.

As used herein, the phrase “functional group” describes a chemical group that has certain functionality and therefore can be subjected to chemical reactions with other components, which reactions typically lead to a bond formation.

As used herein, the term “amine” refers to an —NR′R″ group where R′ and R″ are each hydrogen, alkyl, alkenyl, cycloalkyl, aryl, heteroaryl (bonded through a ring carbon) or heteroalicyclic (bonded through a ring carbon) as defined hereinbelow.

The term “alkyl” as used herein, describes a saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 5 carbon atoms.

As used herein, the term “aldehyde” refers to an —C(═O)—H group.

As used herein, the term “amide” refers to a —C(═O)—NR′— group, where R′ is hydrogen, alkyl, cycloalkyl or aryl.

As used herein, the term “carboxy” refers to an —C(═O)R″ group, where R″ hydroxy, alkoxy, halo and the like.

As used herein, the term “hydroxy” refers to an —OH group.

As used herein, the term “thiol” refers to a —SH group.

As used herein, the term “aminoalkyl” describes an alkyl group, as this term is defined herein, which is substituted at its end-carbon by an amine group, as this term is defined herein.

In a preferred embodiment of the present invention, the second moiety is linked to the first moiety via an amide bond. The amide bond can be formed, for example, between a carboxylic acid of a terminal amino acid residue in the second moiety and a free amine group in the first moiety (e.g., an aminoalkyl, as described to hereinabove). Alternatively, the amide bond can be formed, for example, between an amine group of a terminal amino acid residue in the second moiety and a carboxy group in the first moiety.

As discussed hereinabove, it has been shown that arginine conjugates of oligosaccharides such as aminoglycosides are highly beneficial therapeutic agents. The novel conjugates described herein were designed so as to provide such conjugates with improved performance.

Hence, according to preferred embodiments of the present invention, the first moiety is an oligosaccharide residue, whereby the oligosaccharide is an aminoglycoside antibiotic.

Representative examples of aminoglycoside residues include, without limitation, residues of natural aminoglycoside antibiotics such as, but not limited to, kanamycin, neomycin, seldomycin, tobramycin, kasugamycin, fortimicin, gentamycin, paromomycin, neamine and sisomicin. Alternatively, residues of semi-synthetic derivatives of aminoglycosides such as amikacin, netilmicin and the like can also be used.

Aminoglycosides, by their definition, include one or more free amine groups that can participate in the formation of a covalent bond with a compatible ftnctional group in the second moiety. Mostly, aminoglycosides include both amine groups attached to primary carbons (e.g., aminoalkyl) and amine groups attached to a secondary carbon. As is detailed hereinbelow, the presence of different amine groups and the different reactivity of such amine groups toward week acylating agents, can be beneficially exploited to selectively attach the second moiety at a predetermined position of the aminoglycoside, so as to obtain amino-modified oligo-saccharides.

The second moiety in the conjugates presented herein comprises a plurality of basic amino acid residues. The second moiety can therefore be a polymeric moiety in which two or more units of the polymer are basic amino acid residues.

As mentioned hereinabove, preferably, the second moiety comprises six or more basic amino acid residues.

In a preferred embodiment of the present invention, the second moiety comprises from 6 to 9 basic amino acid residues.

Preferred conjugates according to the present embodiments are composed of a second moiety that comprises 6 or 9 basic amino acid residues.

As used herein, the phrase “basic amino acid residue” describes a residue, as defined herein, of an amino acid that has a pKa value greater than 7. Basic amino acids typically have positively charged side chains at physiological pH due to association with a proton (H⁺). Naturally occurring (genetically encoded) basic amino acids include lysine (Lys, K), arginine (Arg, R) and histidine (His, H), while non-natural (non-genetically encoded, or non-standard) basic amino acids include, for example, ornithine, 2,3,-diaminopropionic acid, 2,4-diaminobutyric acid, 2,5,6-triaminohexanoic acid, 2-amino-4-guanidinobutanoic acid, and homoarginine, as well as derivatives such as D-a-methylarginine, L-α-methylarginine, L-α-methylhistidine, and D-α-methylhistidine.

The phrase “basic amino acid residue” further encompasses residues of L-amino acids and of D-amino acids.

The second moiety therefore includes two or more basic amino acid residues, as described hereinabove, whereby these residues can be the same or different, namely, a combination of various basic amino acid residues. In one embodiment of the present invention, all the basic amino acids in the second moiety are the same and are preferably arginine residues.

In a preferred embodiment of the present invention, the second moiety is a peptide comprising the basic amino acid residues. Such a peptide can further include additional amino acid residues, selected from residues of naturally occurring and non naturally-occurring amino acids. The basic amino acid residues in such a peptide can be either linked one to another or can be interrupted by any of the other amino acid residues.

As used herein, the term “peptide” encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to N-terminus modification (e.g., N-acylation), C-terminus modification, peptide bond modification, including, but not limited to, CH₂—NH, CH₂—S, CH₂—S═O, O═C—NH, CH₂—O, CH₂—CH₂, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH₃)—CO—), ester bonds (—C(R)H—C—O—O—C(R)—N—), ketomethylen bonds (—CO-CH₂—), α-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom.

These modifications can occur at any of the bonds along the peptide chain and even at several (2-3) at the same time.

The peptide, constituting the second moiety according to the preferred embodiment of the present invention, can comprise from 2 to 100 basic amino acid residues, preferably from 2 to 50 basic amino acid residues and more preferably from 2 to 20 basic amino acid residues. Thus, preferably, the peptide constituting the second moiety according to this embodiment can include 2, 3, 4, 5, 6, 7, 8, 9, 10, 11 and up to 20 and even 50 basic amino acid residues.

As is detailed in the Background section above and is further discussed hereinabove, it was shown that oligomers of arginine composed of six or more amino acids, alone or covalently attached to a variety of small molecules, efficiently cross the cell membrane. Thus, in a more preferred embodiment of the present invention, the peptide constituting the second moiety in the conjugates presented herein, comprises at least 6 basic amino acid residues and thus can include 6, 7, 8, 9, 10, 11 and up to 20 and even 50 basic amino acid residues .

Yet further, the present inventors have previously shown that NeoR6 (presented above) was the most efficient anti-HIV-1 compound as compared to other AACs (see, for example, U.S. patent application Ser. No. 10/831224, which is the parent application of the instant application).

It was further shown that positively charged amino acid peptides, in particular poly-arginine, are inhibitors of the co-receptor CXCR4 and thus inhibit infection exclusively by blocking virus-CXCR4 interactions. Specifically, the free 9-arginine-polymer was found to serve as an anti-HIV-1 drug.

Thus, it is particularly preferred embodiments of the present invention, the second moiety is a peptide which comprises from 6 to 9 basic amino acid residues.

According to the presently most preferred embodiment of the present invention, the second moiety is a peptide, which essentially consists of the two or more basic amino acid residues. More preferably, the peptide consists essentially of two or more arginine residues.

Further according to the presently most preferred embodiments of the present invention, the second moiety is a peptide, which essentially consists of 6-9 basic amino acid residues. More preferably, the peptide consists essentially of 6-9 arginine residues. Most preferably, the peptide consists essentially of 6 or 9 basic amino acid residues such as arginine residues.

In another embodiment of the present invention, the basic amino acid residues form a non-peptidic polymeric chain, that is, two or more of the basic amino acid residues are linked one to another via a non-peptidic linker. Thus, for example, two or more of the basic amino acid residues can be linked one to another via a linker such a hydrocarbon chain.

The second moiety can be linked to the first moiety either directly or indirectly. When attached directly, attachment is effected by coupling a functional group of the first moiety with a compatible functional group in the second moiety, as is detailed hereinabove. When attached indirectly, the second moiety can be attached to the first moiety via a spacer such as a hydrocarbon chain. The spacer is selected suitable, namely, having compatible functional groups, for being attached to the first moiety at one end thereof and to the second moiety at another end thereof. Exemplary spacers can include, for example, a hydrocarbon chain having an amine group at one end, which can be coupled to a carboxylic group of a basic amino acid residue and a carboxylic group at another end, which can be coupled to an aminoalkyl of an aminoglycoside.

According to the presently most preferred embodiments of the present invention the first moiety is an aminoglycoside residue, and the second moiety is a peptide consisting essentially of two or more basic amino acid residues such as arginine residues. In particularly preferred embodiments of the present invention, the peptide consists essentially of 6-9 basic amino acid residues such as arginines and more preferably of 6 or 9 basic amino acid residues such as arginine residues. The second moiety is linked to the first moiety via an amide bond, formed between a carboxylic terminus of the peptide and an aminoalkyl group of the aminoglycoside. Representative examples of such compounds are collectively represented in Scheme I hereinabove.

Furthermore, the arginine residues composing the peptide moiety the preferred conjugates presented herein can be residues of either L-arginine and/or of D-arginine.

As is demonstrated in the Examples section that follows, it has been found that conjugates comprising a second moiety that consists essentially of D-arginine residues exhibit a superior performance as anti-viral agents.

However, as is further demonstrated and detailed in the Examples section that follows, it has been found that, depending on the method of preparing the conjugates, conjugates of L-polyarginine and an aminoglycoside such as, for example, neamine and paromomycin, which further comprise an organic residue having a molecular weight of about 55 daltons, can be obtained. These conjugates were found to exhibit a superior performance as compared to other tested L-arginine-containing conjugates. Hence, according to another preferred embodiment of the present invention, a conjugate according to the present embodiments further comprises an organic residue having a molecular weight of about 55 daltons.

While the exact structure of these compounds is still under investigation, it has been established that the surplus weight of these compounds is not a result of metal complexation (e.g., Fe⁵⁵ complexation) (see, the Examples section that follows). Furthermore, as detailed in the Examples section that follows, it has been shown that the 55 daltons residue is an organic residue that is attached to the second moiety (and not to the first moiety). Based on structural studies conducted, it is assumed that this organic residue is attached either to the alpha-amine and/or to the guanidino group of an arginine residue.

As mentioned hereinabove, the present inventors have developed a novel methodology for preparing the conjugates described herein and, particularly, for preparing such conjugates in which the second moiety is attached to a pre-selected position of the first moiety. Thus, according to another aspect of the present invention there is provided a process of preparing the conjugates described hereinabove. The process, according to this aspect of the present invention, is effected by coupling a first compound having at least one saccharide unit and a second compound having two or more basic amino acid residues, preferably in the presence of a coupling agent.

The coupling agent of choice is selected suitable for promoting a reaction between the functional groups at each of the compounds.

Since, as described hereinabove, the first and the second moieties in the conjugate are preferably linked via an amide bond, formed between an amine group (derived from an aminoglycoside) and a carboxylic group (derived from a basic amino acid), preferred coupling agents, according to this aspect of the present invention, include peptide coupling agents.

Representative examples of peptide coupling agents include, without limitation, carbodiimides such as dicyclohexylcarbodiimide (DCC), 1-ethyl-3-(3′-dimethylaminopropyl)carbodiimide (EDC) and N,N′-diisopropylcarbodiimide (DIC); benzotriazoles such as 1-hydroxybenzotriazole (HOBt), 1-hydroxy-56-chlorobenzotriazole (Cl-HOBt) and 1-hydroxy-7-azabenzotriazole (HOAt); and phosphonium and aminium/uronium salts of benzotriazole such as 2-(7-aza-1H-benzotriazole-1-yl)- 1,1,3,3-tetramethyluronium hexafluorophosphate (HATU) benzotriazol-1-yl-N-oxy-tris(pyrrolidino)phosphonium hexafluorophosphate (PyBOP) and 7-azabenzotriazol-1-yl-N-oxy-tris(pyrrolidino)phosphonium hexafluorophosphate (PyAOP).

As mentioned hereinabove and is further discussed in detail in the Examples section that follows, the novel methodology presented herein can be efficiently utilized when the first moiety is derived from a first compound that has an aminoalkyl group. The presence of an aminoalkyl group enables to perform the coupling in high regioselectivity.

Thus, according to preferred embodiments of this aspect of the present invention, at least one saccharide unit in the first compound comprises an aminoalkyl group and the coupling is effected via the aminoalkyl group by first preparing a compound having at least one saccharide unit and at least one aminoalkyl group attached to the saccharide unit, in which any non-alkylamino groups in this compound are protected. Such a compound therefore has one or more aminoalkyl groups, to which the second compound is coupled.

Protecting the non-alkylamino groups can be effected with any of the known and available protecting groups, depending on the chemical nature of the group.

A compound having aminoalkyl groups that are selectively unprotected can be prepared, according to the present embodiments, by selectively protecting the aminoalkyl group with a first protecting group; protecting other functional groups by a second protecting group, being different from the first protecting group; and selectively deprotecting the aminoalkyl group(s) by selective removal of the first protecting group.

Deprotection and protection protocols are well known in the art and the specific procedure is selected according to the protecting groups used.

The selective protection of aminoalkyl is effected by attaching an N-protecting group to the alkylamino group of the saccharide unit. As used herein the phrase “N-protecting group” refers to a chemical group, which is capable of protecting an amino group against undesirable reactions during synthetic procedures. The resulting N-protecting group is derived from an N-protecting compound, which reacts with an amine group, to thereby form the N-protecting group. Similarly, the phrase “protecting group” refers to chemical group which is capable of protecting any functional group against undesirable reactions during synthetic procedures.

The N-protecting compound utilized in the process according to this aspect of the present invention is selected of a spatial size which is suitable for selectively reacting only with the less hindered alkylamino group to thereby selectively protect the alkylamino group of the saccharide unit. In other words, the N-protecting compound is characterized as being a bulky compound, of a relatively large spatial size, as compared with other, typically used N-protecting compounds (e.g., t-Boc).

It will be appreciated that saccharide-containing compounds (referred to herein as the first compound) which are devoid of any amine group can also be used according to this aspect of the present invention. Such compounds can be chemically modified to include such amine groups. For example, an amine group or groups can be attached to saccharide backbones by methods which are well known in the art, such as by azide displacement of saccharide sulphonates or halides, or by a simple conversion of a saccharide hydroxy to amine.

As mentioned hereinabove, the N-protecting group utilized by the present invention has a size suitable for selectively reacting with the alkylamino group and thus, under the synthesis conditions used it will not react with other functional groups of the saccharide unit due to stearic hindrance.

Examples of N-protecting compounds, which can be used in this context of the present invention include, but are not limited to, those disclosed in Greene, “Protective Groups In Organic Synthesis,” (John Wiley & Sons, New York (1981)), which is incorporated by reference as if fully set forth herein.

Preferred N-protecting compounds that are particularly suitable for use in this context of the present invention include, for example, the bulky trityl group (e.g., trityl chloride). Such compounds are advantageous since deprotection of the resulting protecting moiety is effected under very mild reaction conditions, such as in the presence of ytterbium triflate, which does not affect other protected functional groups.

The presently most preferred N-protecting compound is N-(tert-butoxycarbonyloxy)-5-norbornene-endo-2,3-dicarboximide (NBND). As is discussed and demonstrated in the Examples section that follows, this compound has unprecedented selectivity towards aminoalkyls.

In a preferred embodiment of the present invention, the first compound is a saccharide such as a monosaccharide, an oligosaccharide or a polysaccharide, as detailed hereinabove and is preferably an aminoglycoside. Commercially available compounds such as aminoglycosides antibiotics are preferred. Once obtained, the first compound is preferably subjected to the above-described manipulations, so as to provide such a compound having pre-determined reactive groups (e.g., aminoalkyl) available for coupling the second compound.

The second compound can be prepared using any of the known and suitable synthetic procedures, depending on its structure. The basic amino acids composing the second compounds are preferably protected prior to the coupling reaction.

Thus, while preparing the second compound prior to the coupling, preferably, protected amino acids are used, having protected functional groups that are present on the side chains of the amino acids or the carboxylic acid or amine end groups. A variety of protected basic amino acids is commercially available or can be otherwise prepared using known methods. In addition, other functional groups that are present in the second compound can be protected prior to the coupling. The protecting groups protecting the various functional groups in the second compound are collectively referred to herein as a third protecting group.

In cases where the second compound is a peptide, it can be prepared, prior to the coupling, using any of the known methods for peptide syntheses.

Preferably, the peptides are chemically synthesized. Synthetic peptides can be prepared by classical methods known in the art, for example, by using standard solid phase techniques. The standard methods include exclusive solid phase synthesis, partial solid phase synthesis methods, fragment condensation, classical solution synthesis, and even by recombinant DNA technology. See, e.g., Merrifield, J. Am. Chem. Soc., 85:2149 (1963), incorporated herein by reference. Solid phase peptide synthesis procedures are well known in the art and further described by John Morrow Stewart and Janis Dillaha Young, Solid Phase Peptide Syntheses (2nd Ed., Pierce Chemical Company, 1984).

Synthetic peptides can be purified by preparative high performance liquid chromatography (Creighton T. (1983) Proteins, structures and molecular principles. WH Freeman and Co. N.Y.). The composition of the resulting peptide can be confirmed via amino acid sequencing.

Alternatively, the peptides can be isolated from a biological source (e.g., a biological sample) and can be thereafter subjected to protection procedures.

Protein purification methods are well known in the art. Examples include but are not limited to fractionation of samples by ammonium sulfate precipitation and acid or chaotrope extraction. Exemplary purification steps may include hydroxyapatite, size exclusion, FPLC and reverse-phase high performance liquid chromatography. Suitable chromatographic media include derivatized dextrans, agarose, cellulose, polyacrylamide, specialty silicas, and the like. PEI, DEAE, QAE and Q derivatives are preferred. Exemplary chromatographic media include those media derivatized with phenyl, butyl, or octyl groups, such as Phenyl-Sepharose FF (Pharmacia), Toyopearl butyl 650 (Toso Haas, Montgomeryville, Pa.), Octyl-Sepharose (Pharmacia) and the like; or polyacrylic resins, such as Amberchrom CG 71 (Toso Haas) and the like. Suitable solid supports include glass beads, silica-based resins, cellulosic resins, agarose beads, cross-linked agarose beads, polystyrene beads, cross-linked polyacrylamide resins and the like that are insoluble under the conditions in which they are to be used. These supports may be modified with reactive groups that allow attachment of proteins by amino groups, carboxyl groups, sulfhydryl groups, hydroxyl groups and/or carbohydrate moieties. Examples of coupling chemistries include cyanogen bromide activation, N-hydroxysuccinimide activation, epoxide activation, sulfhydryl activation, hydrazide activation, and carboxyl and amino derivatives for carbodiimide coupling chemistries. These and other solid media are well known and widely used in the art, and are available from commercial suppliers. Selection of a particular method is preferably determined by the properties of the chosen support. See, for example, Affinity Chromatography: Principles & Methods, Pharmacia LKB Biotechnology, Uppsala, Sweden, 1988.

The third protecting group can optionally be removed either prior to, concomitant with or subsequent to the coupling, and is preferably effected subsequent to the coupling.

Additional details regarding the process according to this aspect of the present invention can be found in the Examples section that follows. As is demonstrated therein, a variety of conjugates have been successfully prepared using this process, in high yield and purity.

As is further demonstrated in the Examples section that follows, the conjugates described hereinabove have been found highly active in inhibiting HIV infectivity, as well as bacterial infectivity, and, furthermore, were characterized by high levels of cellular intake. As such, these conjugates can be beneficially used in the treatment of e.g., bacterial and viral infections, and in the preparation of medicaments for treating such medical conditions.

Thus, according to a further aspect of the present invention there is provided a method of treating a medical condition associated with an infectious microorganism. The method is effected by administering to a subject in need thereof a therapeutically effective amount of a conjugate as described hereinabove.

As used herein, the term “treating” refers to alleviating or diminishing a symptom associated with a bacterial or viral infection. Preferably, treating cures, e.g., substantially eliminates, the symptoms associated with the infection and/or substantially decreases the bacterial or viral load in the infected tissue.

Preferred individual subjects according to the present invention are mammals such as canines, felines, ovines, porcines, equines, bovines, humans and the like.

Preferred medical conditions that are treatable by the method according to this aspect of the present invention include conditions caused by an infectious microorganism such as a virus or a bacterial strain and therefore typically include viral and bacterial infections.

The conjugates presented herein enable treatment of bacterial infections even in cases where such infections are resistant to conventional antibiotic agents, or when toxicity of conventional antibiotics prevents utilization of an aggressive treatment regimen. Thus, in a preferred embodiment, the medical condition is caused by a resistant bacterial strain.

Although the complete mechanism of action of these conjugates is not thoroughly understood, it is conceivable that they interfere with bacterial targets i.e., RNA-protein complexes (RNP), thus blocking various biological processes necessary for pathogen growth and proliferation (for further details see Eubank et al. (2002) FEBS Lett. 511: 107-112).

Bacterial infections treated according to the present invention include opportunistic aerobic gram-negative bacilli such as the genera Pseudomonas, bacterial infection caused by P. aeruginosa, bacterial infections caused by gram-positive bacilli such as that of the genus Mycobacterium, and mycobacteria, which causes tuberculosis-like diseases.

Thus, examples of resistant bacterial strains include gram negative strains such as, but not limited to, various strains of Escherichia coli, Pseudomonas aeruginosa, Klebsiella pneumoniae, Proteus mirabilis, Acinetobacter baumannii, Moraxella catarrhalis, Serratia marcescens, Enterococcus Faecalis, Enterobacter cloacae and Enterobacter aerogenes (for specific examples, see Tables 6-8 in the Examples section that follows), and various strains of Gram positive strains such as, but not limited to, Staphylococcus aureus, Staphylococcus epidermidis, Streptococcus pyogenes, Streptococcus bovis, Streptococcus Pneumoniae, Streptococcus Pyogenes, Streptococcus Agalactiae, Bacillus subtilis, Enterococcus faecalis, Enterococcus faecium and Listeria monocytogenes (for specific examples, see Tables 6-8 in the Examples section that follows).

Viral infections which can be treated using the conjugates presented herein include but are not limited to HIV infections, infections caused by the equine infectious anemia virus (EIAV) (Litovchick et al. (2000) supra) and hepatitis C viral infections. Also included are AIDS and AIDS manifestations such as, for example, Kaposi sarcoma.

As is further demonstrated on the Examples section that follows, the conjugates presented herein exhibited high affinity to CXCR-4. These conjugates can therefore be further utilized in treating disorders which involve disregulated (e.g., upregulated) function of the chemokine receptor CXCR-4 and/or its cognate ligand SDF-1α.

CXCR4 plays an important role in many biological functions, such as B-cell lymphopoiesis, neuronal cell migration and vascular development (Nagasawa et al. (1996) Nature 382, 635-638; Ma et al. (1998) Proc. Natl. Acad. Sci. U. S. A 95, 9448-9453; Zou et al. (1998) Nature 393, 595-599). The stromal cell-derived factor-1 (SDF-1α), the only known natural ligand of CXCR4, displays important roles in migration, proliferation and differentiation of leukocytes (Bleul et al., 1996; Oberlin et al., 1996).

Disorders which involve abnormal function of CXCR-4 include but are not limited to cancer such as metastatic cancer in which transedothelial cell migration plays a central role in disease progression (Mohle Ann N Y Acad Sci. (1999) Apr. 30;872:176-85).

The conjugates utilized in the method according to this aspect of the present invention can be administered via any administration route, including, but not limited to, the oral, rectal, transmucosal, intestinal, parenteral, intramuscular, subcutaneous, intrathecal, direct intraventricular, intravenous, inrtaperitoneal, intranasal, or intraocular routes, as well as by inhalation.

The conjugates utilized in the method according to this aspect of the present invention can be further utilized in combination with other therapies. Thus, for example, the conjugates can be co-administered, either simultaneously or separately, during the treatment period, with another antibacterial or antiviral agent.

The conjugates presented herein can be administered or otherwise utilized either per se, or as part of a pharmaceutical composition where it is mixed with a pharmaceutically acceptable carrier.

Hence, according to an additional aspect of the present invention there is provided a pharmaceutical composition, which comprises any of the conjugates described hereinabove and a pharmaceutically acceptable carrier.

As used herein a “pharmaceutical composition” refers to a composition of one or more of the conjugates described herein (referred to hereinafter as an active ingredient), or physiologically acceptable salts or prodrugs thereof, with other chemical components such as physiologically suitable carriers and excipients. The purpose of a pharmaceutical composition is to facilitate administration of a compound to an organism.

Hereinafter, the phrases “pharmaceutically acceptable carrier” and “physiologically acceptable carrier” are used interchangeably to refer to a carrier or a diluent that does not cause significant irritation to a treated individual and does not abrogate the biological activity and properties of the active ingredient.

Herein the term “excipient” refers to an inert substance added to a pharmaceutical composition to further facilitate administration of active ingredients. Examples, without limitation, of excipients include calcium carbonate, calcium phosphate, various sugars and types of starch, cellulose derivatives, gelatin, vegetable oils and polyethylene glycols.

Techniques for formulation and administration of the pharmaceutical compositions of the present invention may be found in “Remington's Pharmaceutical Sciences,” Mack Publishing Co., Easton, Pa., latest edition, which is incorporated herein by reference.

Suitable routes of administration may, for example, include oral, rectal, transmucosal, intestinal or parenteral delivery, including intramuscular, subcutaneous and intramedullary injections as well as intrathecal, direct intraventricular, intravenous, inrtaperitoneal, intranasal, or intraocular injections.

Alternately, one may administer a pharmaceutical composition in a local rather than systemic manner, for example, via injection of the composition directly into the area of infection often in a depot or slow release formulation, such as described below.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredient into compositions which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art.

For oral administration, the pharmaceutical composition can be formulated by combining the active agents with pharmaceutically acceptable carriers well known in the art. Such carriers enable the pharmaceutical composition used by the method of the invention to be formulated as tablets, pills, dragees, capsules, liquids, gels, syrups, slurries, suspensions, and the like, for oral ingestion by a patient. Pharmacological compositions for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose compositions such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active ingredient doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the agents for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insulator may be formulated containing a powder mix of the active ingredient and a suitable powder base such as lactose or starch.

Ophthalmic formulations, eye ointments, powders, solutions and the like, are also contemplated as being within the scope of this invention.

The compositions described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active ingredient in water-soluble form. Additionally, suspensions of the active ingredient may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or formulations, which increase the solubility of the active ingredient to allow for the composition of highly concentrated solutions.

Alternatively, the active ingredient may be in powder form for constitution with a suitable vehicle, e.g., sterile, pyrogen-free water, before use.

The composition of the present invention may also be formulated in rectal compositions such as suppositories or retention enemas, using, e.g., conventional suppository bases such as cocoa butter or other glycerides.

In addition to the formulations described previously, a composition of the present invention may also be formulated for local administration, such as a depot composition. Such long acting formulations may be administered by implantation (for example subcutaneously or intramuscularly) or by intramuscular injection. Thus, for example, the composition may be formulated with suitable polymeric or hydrophobic materials (for example, as an emulsion in an acceptable oil) or ion exchange resins, or as sparingly soluble derivatives such as sparingly soluble salts. Formulations for topical administration may include, but are not limited to, lotions, suspensions, ointments gels, creams, drops, liquids, sprays emulsions and powders.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose. More specifically, a therapeutically effective amount means an amount of active ingredient effective to prevent, alleviate or ameliorate symptoms of disease or prolong the survival of the subject being treated.

Determination of a therapeutically effective amount is well within the capability of those skilled in the art.

For any composition used by the methods of the invention, the therapeutically effective amount or dose can be estimated initially from cell culture assays and cell-free assays (See the Examples section, which follows). For example, a dose can be formulated in animal models to achieve a circulating concentration range that includes the IC₅₀ as determined in in-vitro assays. Such information can be used to more accurately determine useful doses in humans.

Regardless, toxicity and therapeutic efficacy of the pharmaceutical compositions described herein can be determined by standard pharmaceutical procedures in experimental animals, e.g., by determining the IC₅₀ and the LD₅₀ (lethal dose causing death in 50% of the tested animals) for a subject ingredient. The data obtained from assays can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al.(1975), in The Pharmacological Basis of Therapeutics, Ch. 1 p. 1).

Dosage amount and interval may be adjusted individually to provide plasma levels of the active ingredient, which are sufficient to maintain the required effects, termed the minimal effective concentration (MEC). The MEC will vary for each composition, but can be estimated from in vitro data; e.g., the concentration necessary to achieve 50-90% inhibition (see Example 1 of the Examples section). Dosages necessary to achieve the MEC will depend on individual characteristics and route of administration. HPLC assays or bioassays can be used to determine plasma concentrations.

Dosage intervals can also be determined using the MEC value. Compositions should be administered using a regimen, which maintains plasma levels above the MEC for 10-90% of the time, preferable between 30-90% and most preferably 50-90%.

It is noted that, in the case of local administration or selective uptake, the effective local concentration of the drug may not be related to plasma concentration. In such cases, other procedures known in the art can be employed to determine the effective local concentration.

Depending on the severity and responsiveness of the infection to be treated, dosing can also be a single administration of a slow release composition, with course of treatment lasting from several days to several weeks or until cure is effected or diminution of the infection state is achieved.

The amount of a composition to be administered will, of course, be dependent on the subject being treated, the severity of the infection, the manner of administration, the judgment of the prescribing physician, etc.

Compositions of the present invention can be packaged in a dispenser device, as one or more unit dosage forms as part of an FDA approved kit, which preferably includes instruction for use, dosages and counter indications. The kit can include, for example, metal or plastic foil, such as a blister pack suitable for containing pills or tablets, or a dispenser device suitable for use as an inhaler. The kit may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising an active ingredient suitable for use with the present invention may also be prepared, placed in an appropriate container, and labeled for treatment of a medical condition, as described hereinabove.

The pharmaceutical composition can further comprise an additional antibacterial or antiviral agent.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

### CHEMICAL SYNTHESES

Materials and Analytical Methods:

All commercially available chemicals were reagent grade and were used without further purification.

Paromomycin and neomycin sulphates were purchased from Sigma Chemical Co.

All aminoglycosides were used as a free base. The corresponding ammonium salts were converted to the free base using Amberlite IRA 400 (OH⁻) ion-exchange resin.

N-hydroxy-5-norbornene-endo-2,3-dicarboximide (Aldrich), di-tert-butyl dicarbonate (Merck), thallous ethoxide (Aldrich), benzylchloroformate (CbzCl), 1-hydroxybenzotriazole (HOBT), N-methylmorpholine (NMM), benzyloxycarbonyl-Arginine (NO₂)—OH, palladium charcoal (10%) (Fluka), N-(3-dimethylaminopropyl)-N′-ethylcarbodiimide hydrochloride (EDC) (Aldrich), Wang resin (100-200 mesh, Novabiochem, Switzerland), Fmoc (3,9-Fluorenylmethoxycarbonyl)-Arg(Pbf (2,2,4,6,7-pentamethyldihydrobenzofurane-5-sulfonyl))-OH and benzotriazole-1-yl-oxy-tris-pyrrolidino-phosphonium hexafluorophosphate (PyBOP) (Novabiochem, Switzerland) were used without any further purification.

Neamine hydrochloride was prepared from neomycin sulphate by methanolysis as previously described (see, J. Am. Chem. Soc. (1952) 74, 3420), with a slight modification based on Grapsas et al. (J. Org. Chem. (1994) 59, 1918).

Column chromatography was conducted using Merck silica gel (Kieselgel 60 (0.063-0.200 mm)).

Analytical Thin-layer chromatography (TLC) was performed with 0.2 mm silica-coated aluminium sheets, visualization by UV light or by spraying an aqueous solution of ninhydrin (0.25%) and then heating the plate.

Analytical RP-HPLC was conducted using an E040720-5-1 Vydac C18 column, at a flow rate of 1 ml/minute at 220, 230 and 280 nm, with a 5-65% linear acetonitrile gradient in 0.1% aqueous trifluoroacetic acid (TFA) over 30 minutes. Preparative RP-HPLC used an E040519-4-4 Vydac C18 column, at a flow rate of 3 ml/minute at 220, 230 and 280 nm, with a 5-65% linear acetonitrile gradient in 0.1% aqueous TFA over 30 minutes. The major HPLC peak was collected and further identified by mass spectroscopy (MALDI-TOF).

Preparation of N-(tert-Butoxycarbonyloxy)-5-norbornene-endo-2,3 dicarboximide (NBND): NBND was chosen as a reagent for its unprecedented selectivity towards different amines, which makes it ideally suited for application to aminoglycoside chemistry. Particularly, NBND was selected as a weak acylating agent, toward which hindered and unhindered amino groups have different reactivity. The extent of selectivity shown by NBND is unprecedented, which makes this reagent ideally suited for application to aminoglycoside chemistry. Upon reaction of NBMD with an amine group, a “Boc” protecting group is actually obtained. Such a protecting group can be easily removed prior to the following conjugation reaction.

NBND was prepared as previously described and shown in Scheme 2 below, using N-hydroxy-5-norbomene-endo-2,3-dicarboximide and di-tert-butyl dicarbonate in the presence of thallous ethoxide (Grapsas, I.; Cho, Y. J.; Mobashery, S. (1994), J. Org Chem., 59, 1918).

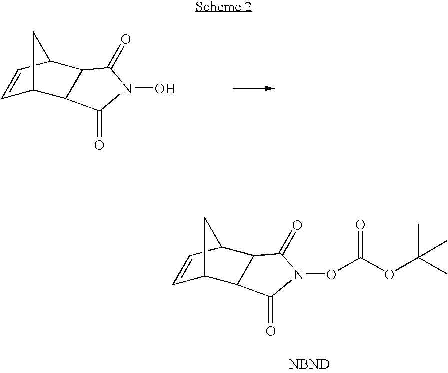

Preparation of Selectively Protected Aminoglycosides—General Procedure:

In order to efficiently and selectively conjugate polyarginine-containing moieties to aminoglycosides, regioselective, functionalized aminoglycosides were prepared. Since each aminoglycoside has several primary amines of approximately comparable reactivity, a special synthetic pathway was designed, based on the difference in reactive of amino groups at primary carbons and those at secondary carbons. Indeed, within several primary amino groups, one amino group of the neamine and paromomycin and two amino groups of neomycin are at primary carbons, the remaining primary amines of the three aminoglycosides are at secondary carbons. These primary amino groups which are at primary carbons are relatively more reactive than the other amino groups.

While a direct reaction between polyarginine and aminoglycosides (neamine, paromomycin and neomycin) resulted in an inseparable complex mixture, a longer synthesis route was employed, involving: (i) a selective introduction of the temporary protecting group to the amine(s) which are at methylene (primary) carbon(s) by an efficient selective acylating agent (e.g., NBND); (ii) protection of the remaining amine functions; and (iii) deprotection of the temporary protecting group(s) from the amino group(s) which are at methylene carbons. The resulting regioselective finctionalized aminoglycoside was then utilized for preparing the conjugate, as described hereinbelow.

The protection and de-protection of the various amino group sites on the aminoglycosides was conducted based on published procedures as follows: Regioselective tert-butoxycarbonyl (BOC) protective groups (obtained from NBND) were use to selectively protect unhindered amino group sites (attached to primary carbon(s)) of the aminoglycosides, based on Grapsas (1994, supra); on Grapsas et al. (2001, Arch. Pharm. Pharm. Med. Chem., 334, 295); and on Sainlos et al. (2003, Eur. J. Org Chem., 2764). Protection of the remaining amino groups was achieved using known procedures (V. Kumar and W. A. Remers, (1978), J. Org. Chem., 43, 3327). Deprotection of BOC protecting groups was conducted using TFA.

Using the above procedure, exemplary compounds according to the present invention have been prepared, as follows:

Preparation of Selectively Protected Neamine (Compound 1α), Paromycin (Compound 2a) and Neomycin (Compound 3a):

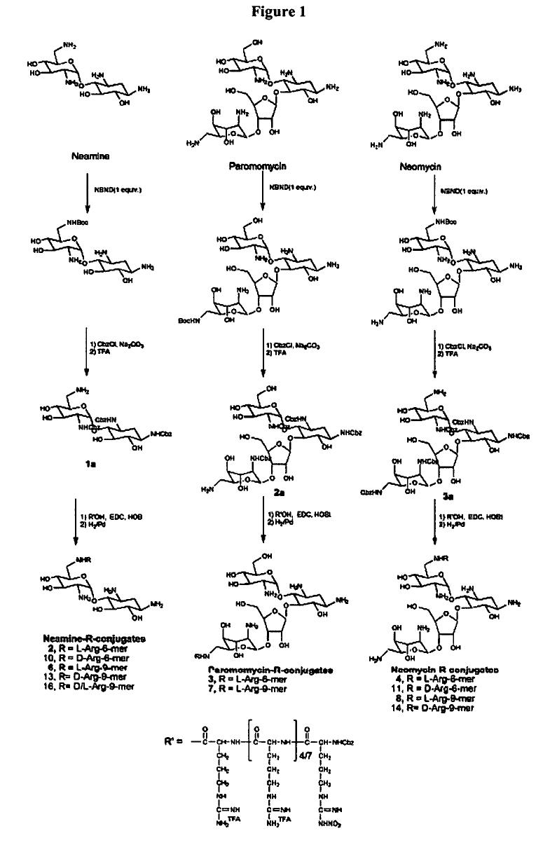

The overall synthetic pathway for preparing Compounds 1a, 2a and 3a is presented in FIG. 1. The free base aminoglycosides (neamine, paromomycin or neomycin) were dissolved in dioxane:water (1:1, v/v), triethylamine (1.5 equivalents) was added and the resulting solution was stirred for 10 minutes. NBND (1 equivalent, prepared as described hereinabove) was then added in one portion and the mixture was stirred at room temperature for approximately 15 hours. TLC analysis of the reaction mixture, using a mixture of 4:2:1 n-BuOH/AcOH/H₂O as eluent, showed the presence of one major product, which was proved to be the desired monocarbamoylated product, with traces of the starting material and polycarbamoylated products. In the case of neomycin, a mixture of two mono-Boc-neomycin derivatives (at ring I and IV) is possible. Mass spectrum analysis confirmed receiving the mono-Boc-neomycin derivative (see Table 1 below for the MS data obtained for the corresponding final product).

The solvents were thereafter removed under reduced pressure and the residue was dissolved in water and washed with ethyl acetate (3×25 ml). The aqueous layer was evaporated under reduced pressure and the residue was dissolved in an acetone:water mixture (7:3, v/v). Sodium carbonate (1.5 equivalents, for each free amino group) was then added and the solution was cooled to 0° C. Benzylchloroformate (1.5 equivalents, for each free amino group) in acetone was added drop wise to the solution and the resulting mixture was stirred at 0° C. for 2 hours and then left at room temperature for 15 hours. TLC analysis of the reaction mixture (using a mixture of 8.5:1.5 CH₂Cl₂/MeOH as eluent) proved complete conversion of the starting material to the desired compound.

Solvents were then removed under reduced pressure, and the residue was extracted three times with warm ethyl acetate. The ethyl acetate layer was washed twice with water, dried over sodium sulphate, concentrated and the residue was consecutively mixed with ether (which was then separated by decantation) so as to obtain a white solid. The solid material was dissolved in dichloromethane and treated with TFA (10% v/v) at room temperature for 3 hours. The solution was thereafter concentrated and the residue was resuspended in ether. Ether was decanted and a white material was obtained. Column chromatography (silica gel, using a mixture of 8.4:1.4:0.2 CH₂Cl₂/MeOH/NH₄OHas eluent) afforded the pure Compounds 1a, 2a and 3a.

MS (MALDI-TOF) Compound 1a: m/z=747.176 (M+Na) (calculated: 747.759).

MS (MALDI-TOF) Compound 2a: m/z=1152.61 (M⁺) (calculated: 1152.163).

MS (MALDI-TOF) Compound 3a: m/z=1307.391 (M+Na) (calculated: 1307.302).

Preparation of Polyarginine Peptides—General Procedure:

Polyarginine peptides, composed of L, D or L/D arginine residues, were synthesized manually by standard solid phase peptide synthesis technique using polystyrene-1%-divinylbenzene-based Wang resin containing Fmoc-protected (L or D) arginine residue. The resin was swelled in N-methyl-2-pyrrolidinone (NMP) for 30 minutes and was then washed five times with dimethylformamide (DMF), twice mixing it with a solution of 20% piperidine in DMF for 15 minutes. After washing with DMF (4 times) and dichloromethane (DCM) (twice), the resin was finally tested for ninhydrin (Kaiser test). Coupling was conducted with Fmoc-L or D-Arg(Pbf)-OH (2 equivalents) and PyBOP (2 equivalents) in the presence of N-methylmorpholine (four equivalents) in DMF for 60 minutes. Completion of the reaction was confirmed by ninhydrin test. This step was repeated until the desired length of the polymer was obtained. Final coupling was conducted with Cbz-Arg(NO₂)—OH or acetic acid (for N-terminal acetylated peptides) for both 6- and 9-mers of arginine. This step was necessary to keep the N-terminal blocked during trifluoroacetic acid (TFA) cleavage of the peptide from the resin.

The resin was then drained and washed consecutively with DMF (4 times), DCM (4 times) and methanol (4 times), and was thereafter dried under vacuum. The peptides were cleaved from the resin by mixing with a TFA:triethylsilane:water (90:5:5) mixture for 12 hours. The long reaction time was required to assure the complete removal of the Pbf protecting groups from the arginine polymers. The peptides were subsequently filtered from the resin and washed with TFA (twice). The combined filtrates were then concentrated under reduced pressure to half of the volume, precipitated using cold diethyl ether, purified by HPLC and verified by mass spectroscopy (MALDI-TOF).

Preparation of L-Arg-6mer-NH₂ (Compound 1), L-Arg-9-mer-NH₂ (Compound 5), D-Arg-6-mer-NHA4c (Compound 9), D-Arg-9-mer-NHAc (Compound 12), D/L-Arg-9-mer-NH₂ (Compound 15) and L-Arg-9-mer-NHA4c (Compound 17):

Using the above general procedure, exemplary compounds according to the present invention, as presented above, have been prepared.

Table 1 below presents the structures and MS data of the various 6- and 9-oligomers of L, D and L/D arginine residues (see, Compounds 1, 5, 9, 12, 15 and 17). The purity of all compounds was around 95%, as determined by HPLC analysis and proven by mass spectroscopy (MALDI-TOF).

Preparation of HO-Arg(NO₂)-(Arg(NO₂)Arg(NO₂)—NH-Cbz (Arg(NO2)-6-mer): Boc-Arg(NO₂)—OH was mixed with N-hydroxysuccinimide (1.1 equivalents) in DMF, the solution was cooled to −5-10° C., and dicyclohexylcarbodiimide (DCC) (1.1 equivalents) was added thereto. The solution was maintained at this temperature for 2 hours. A solution of H-Arg(NO₂)—OH (1.1 equivalents) and NaHCO₃ (2 equivalents) in water was then added and the resulting mixture was stirred at room temperature for 4 hours. The solvents were thereafter removed under reduced pressure, and the residual mixture was cooled to about 0° C. and acidified with 0.1 N H₂SO₄. The mixture was dissolved in dichloromethane, and washed with water and saline. The organic layer was then separated, dried over sodium sulphate and concentrated. The residue was then treated with trifluoroacetic acid for 30 minutes at room temperature, and the solvent was thereafter evaporated in vacuum. This cycle was repeated 4 times. The final coupling was conducted with Cbz-Arg(NO₂)—OH to afford the desired compound.

Preparation of HO-(Arg(NO₂))₂—NH-Cbz (Arg(NO₂)-2-mer): Boc-Arg(NO₂)—OH was mixed with N-hydroxysuccinimide (1.1 equivalents) in DMF, the solution was cooled to −5-10° C., and dicyclohexylcarbodiimide (DCC) (1.1 equivalents) was added thereto. The solution was maintained at this temperature for 2 hours. Then Cbz-Arg(NO₂)—OH was added to the reaction mixture. The solvents were thereafter removed under reduced pressure, and the residual mixture was cooled to about 0° C. and acidified with 0.1 N H₂SO₄. The mixture was dissolved in dichloromethane, and washed with water and saline. The organic layer was then separated, dried over sodium sulphate and concentrated. The residue was then treated with trifluoroacetic acid for 30 minutes at room temperature, and the solvent was thereafter evaporated in vacuum, to afford the desired compound.

Preparation of Polyarginine Conjugated Aminoglycosides (PAACs)—General Procedure:

The coupling of the polyarginine peptides (6- and 9- mers) with the selectively functionalized aminoglycosdies was conducted by using EDC as a coupling reagent in the presence of HOBT and diisopropylethylamine (DIEA). The final pAACs were obtained by deprotecting the remaining protecting groups (Cbz and NO₂) via hydrogenation in the presence of Pd/C.

Thus, to a cooled solution of a protected aminoglycoside, prepared as described above, in DMF, diisopropylethylamine (DIEA), a polyarginine, prepared as described above, and HOBT were added, followed by slow addition of EDC. The mixture was stirred at room temperature for approximately 15 hours and was thereafter concentrated under reduced pressure. The residue was washed with sodium bicarbonate and water to remove any excess of the polypeptide, HOBT or EDC. The resulting material was dissolved in a dioxane:ethanol:water:acetic acid (1:1:1:1, v/v/v/v) mixture, 10% palladium-charcoal catalyst (15% w/w) was added and the resulting mixture was subjected to hydrogenation at atmospheric pressure overnight. The catalyst was thereafter removed by filtration, and the filtrate was concentrated under reduced pressure. The residue was precipitated from acetone and was re-crystallized from an ethanol/acetone mixture, so as to afford the polyarginine-conjugated aminoglycosides.

Preparation of L-Arg-6-mer-neamine (Compound 2), L-Arg-9-mer-neamine (Compound 6), L-Arg-6-mer paromycin (Compound 3), L-Arg-9-mer-paromycin (Compound 7), L-Arg-6-mer-neomycin (Compound 4), L-Arg-9-mer-neomycin (Compound 8), D-Arg-6-mer-neamine-NHAc (Compound 10), D-Arg-9-mer-neamine-NHAc (Compound 13), D-Arg-6-mer-neomycin (Compound 11), D-Arg-9-mer-neomycin (Compound 14), D/L-Arg-9-mer-neamine (Compound 16) and L-Arg-2-mer-neamine (Compound 21): To a cooled solution of Compound la, 2a or 3a, prepared as described above, in DMF, diisopropylethylamine (DIEA, 1.2 equivalents), an arginine dimer (Arg(NO₂)-2-mer), hexa- (Arg-6-mer) or nona- (Arg-9-mer) peptide, prepared as described above, and HOBT (1.5 equivalents) were added, followed by a slow addition (during 2 hours) of EDC (1.5 equivalents). The resulting mixture was stirred at room temperature for approximately 15 hours and was thereafter concentrated under reduced pressure. The residue was washed with 0.2 M sodium bicarbonate and water, to remove any excess of the peptides, HOBT or EDC. During the conjugation, no interference of the guanidinium headgroups of arginine was observed during conjugation, as expected from the strongly basic character and tight association of the guanidinium ions with TFA counterions (Litovchick et al. (2001) supra; Lapidot (2004) supra). It appears that the tertiary amine DIEA is not sufficiently basic to deprotonate the guanidinium headgroup.

The resulting material was dissolved in a dioxane:ethanol:water:acetic acid (1:1:1:1, v/v/v/v) mixture and a 10% palladium-charcoal catalyst (15% w/w) was added. The mixture was subjected to hydrogenation at atmospheric pressure overnight. The catalyst was then removed by filtration, and the filtrate was concentrated under reduced pressure. The oily residue was precipitated with acetone and was recrystallized with ethanol/acetone to give the product as a white powder.

Final purification was conducted by HPLC (to afford 95% purity of the product, see above). TFA counterions were neutralized using Amberlite IRA 400 (OH⁻) ion-exchange resin and the product was converted into acetate salt, liophilized and characterized by MALDI-TOF mass spectrometer.

Table 1 below presents the structures and MS data of the various polyarginine-neamine, paromomycin and neomycin conjugates prepared. Set A presents conjugates containing L-arg-mers; Set B presents conjugates containing D-Arg-mers; and set C presents conjugates containing D/L-Arg-mers. In the case of neomycin, conjugates were obtained as a 1:1 mixture of two neomycin conjugates, in which either ring I or IV was conjugated to the arginine chain.

¹H NMR (500 MHz, D₂O) analysis of L-Arg-6-mer-neamine (Compound 2) revealed the presence of the characteristic groups of the arginine moieties at chemical shifts (6) of 3.54 (H_{α}) and 3.23 ppm (H_{β}) as well as at 1.6 and 1.8 ppm (H_{γ, δ}). The characteristic neamine proton signals were observed at 1.28, 1.67 and 3.0 to 3.5 (overlapping multiplet) ppm.

¹³C- NMR (D₂O) analysis of L-Arg-6-mer-neamine (Compound 2) revealed carbon resonances of the C-arginine amide moieties at 26.08, 26.53, 33.14 and 51.44 ppm, as well as carbon resonances of the neamine at 32.81, 43.21, 51.61, 51.73, 55.72, 71.89, 72.00, 73.45, 75.83, 76.02, 84.84, 99.09, 159.77 (guanidinium carbon) and 184.10 (amide carbons).

TABLE 1

| Compound | | | Aminoglycoside | MS (m/z) | |
|---|---|---|---|---|---|
| No. | | Peptide/Conjugate | (Amg) | Calculated | Observed |
| A | 1 | HO-RRRRRR-NH₂ | — | 955.142 | 956.231 |
| | 2 | Amg-RRRRRR-NH₂ | Neamine | 1259.486 | 1259.763 |
| | 3 | Amg-RRRRRR-NH₂ | Paramomycin | 1552.756 | 1552.812 |
| | 4 | Amg-RRRRRR-NH₂ | Neomycin | 1551.772 | 1551.431 |
| | 5 | HO-RRRRRRRRR-NH₂ | — | 1423.705 | 1423.987 |
| | 6 | Amg-RRRRRRRRR-NH₂ | Neamine | 1728.039 | 1728.85 |
| | 7 | Amg-RRRRRRRRR-NH₂ | Paramomycin | 2021.309 | 2022.31 |
| | 8 | Amg-RRRRRRRRR-NH₂ | Neomycin | 2020.325 | 2020.83 |
| B | 9 | HO-rrrrrr-NHAc | — | 997.178 | 997.61 |
| | 10 | Amg-rrrrrr-NHAc | Neamine | 1301.523 | 1301.80 |
| | 11 | Amg-rrrrrr-NHAc | Neomycin | 1593.809 | 1594.14 |
| | 12 | HO-rrrrrrrrr-NHAc | — | 1465.742 | 1466.00 |
| | 13 | Amg-rrrrrrrrr-NHAc | Neamine | 1770.076 | 1770.43 |
| | 14 | Amg-rrrrrrrrr-NHAc | Neomycin | 2062.362 | 2063.00 |
| C | 15 | HO-RRrRrRrRR-NH2 | — | 1423.705 | 1424.812 |
| | 16 | Amg-RRrRrRrRR-NH2 | Neamine | 1728.039 | 1728.514 |
| | 17 | HO-RRRRRRRRR-NHAc | — | 1465.710 | 1466.13 |

R: L-agrinine;
r: D-arginine;
Amg: aminoglycoside as detailed in the third column.

Preparation of Neam-R6 55 (N55) and Paromo-R6 55 (P55): Two compounds were prepared as described above, using the peptide HO-Arg(NO₂)-(Arg(NO₂))₄-Arg(NO₂)—NH-Cbz, prepared as described above, in the conjugation to the aminoglycosides. Interestingly, using a different hexapeptide resulted in conjugates of neamine or paromomycin which showed a mass surplus of 55 Daltons. These conjugate were therefore termed N55 and P55, respectively.

The ¹H- and ¹³C-NMR of Compound N55 showed few additional signals apart from the signals of the corresponding PAACs. A mass spectrum of this compound showed a mass unit of 1259.76 (calculated 1259.48). Furthermore, there were three signals at M+55 (1314.79), M+110 (1369.83), M+165 (1424.81) of almost the same intensities.

It is important to note that N55 and P55 were obtained only while using the L-amino acids, and when the specific method of preparing the polyarginines, as detailed above, was used.

While the exact structure of these compounds is still under investigation, it has been established, by atomic absorption measurements, that both N55 and P55 are not formed as a result of metal complexation (data not shown). In particular, the possibility that the 55 Daltons-residue is Iron (Fe⁵⁵) has been eliminated.

Furthermore, based on 2D-NMR studies and the elemental analysis detailed above, it has been concluded that the 55 daltons-residue is an organic residue. It has been further concluded that this organic residue is attached to the alpha-amine and/or to the guanidino group of an arginine residue, and is not attached to the antibiotic moiety.

### ACTIVITY ASSAYS

Materials and Experimental Methods:

Cell Cultures:

MT2 cells (lymphocyte cell line permissive to T-tropic HIV-1 isolates) were cultured in RPMI 1640, containing 10% fetal calf serum (FCS) and antibiotics.

cMAGI HIV-1 reporter cells were cultured in DMEM, containing 10% FCS and antibiotics (100 μg/ml penicillin; 100 μg/ml streptomycin; 0.25 μg/ml fungizone; 200 μg/ml G418; 100 μg/ml hygromycin B; 1 μg/ml puromycin).

Cytotoxicity:

Cytotoxicity of the various pAACs was determined using the trypan blue exclusion assay. In brief, a sample of the cell suspension was diluted 1:1 (v/v) with 0.4% trypan blue and the cells were counted using a hemocytometer. Results are expressed as the percentage of dead cells.

Cell Inhibition of HIV-1 Replication:

Inhibition of HIV-1 cell replication was tested using the cMAGI assay, which is based on the ability of HIV-1 TAT to transactivate the expression of an integrated β-galactosidase reporter gene driven by the HIV-LTR (B. Chackeria et al., J. Virol., (1997) 71, 3932; Collins et al., Nat. Med. (2000) 6, 475). The β-galactosidase reporter is modified to remain localized in the nucleus where it can be detected with the X-gal (5-bromo-4-chloro-3indoyl-β-D-galactopyranoside) substrate as an intense nuclear stain within a few days of infection.

HIV-1 isolates were propagated by subculture in MT2 cells (Litovchick et al. (2001) supra). Aliquots of cell-free culture supernatants were used as viral inoculum. PAACs were dissolved in the RPMI 1640 medium. Cytotoxicity determinations were carried out in MT2 cells by trypan blue exclusion assay. Viral inhibition was determined by incubating MT2 or cMAGI HIV-1 reporter cells with 0.2-0.5 multiplicity of infection of HIV-1 wild type or resistant virus for 4 days at 37° C. in the presence or absence of various concentrations of pAACs. HIV-1 infection of cMAGI cells was determined by counting the number of HIV-1-infected cells (stained blue). The cytopathic effects of the viral infection of MT2 cells were also analyzed by microscopic assessment of syncytium formation.

Hemolytic Activity:

Human erythrocyte (HRBC) suspension with EDTA was rinsed 3 times with PBS by centrifugation for 10 minutes at 2000 RPM and resuspended in PBS. The tested compounds (hexa-arginine and nona-arginine conjugates of neamine, paromomycin and neomycin) in PBS solution were serially diluted in 96 well round bottom plate and then 50 μl of hRBC suspension were added to reach a final volume of 100 μl (final erythrocyte concentration 4% v/v and final compounds concentrations were 1.56-100 μM). The resulting suspensions were agitated for 60 minutes at 37° C. The samples were then centrifuged at 2000 RPM for 10 minutes. Supernatant from all the wells were then transferred to another 96 well flat bottom plate and the release of hemoglobin was monitored by measuring the absorbance at 540 nM. Controls for zero hemolysis and 100% hemolysis consisted of hRBC suspended in PBS and Triton 1%, respectively. All the tested compounds have not revealed any hemolytic activity up to 100 μM

Fluorescent Probes:

pAACs-fluorescein isothiocyanate: The acetate counter-ions of the pAACs and of the oligopeptides alone were first removed by Amberlite IRA 400 (OH⁻) ion-exchange. Then fluorescein 5(6)-isothiocyanate (FITC, Sigma) was added in water:methanol:dioxane (1:1:1, v/v/v) medium in the presence of 2 equivalents of triethyl amine, and was mixed for 2 hours at room temperature. After removal of the solvents under reduced pressure the FITC derivatives were purified by extraction with absolute ethanol, and were then converted to the corresponding acetate salts.

Cellular uptake using pAACs and polyarginine peptide-fluorescent derivatives: cMAGI cells were incubated in a 8-well plate (2000 cells/well) with a pAACs-FITC or a peptide-FITC derivative at a final concentration of 5 and 15 μM, for 30 minutes at 37° C. After incubation, cells were washed 3 times with phosphate buffered saline 3 times and were subjected to confocal microscopy measurements (Olympus IX70 FV500 confocal laser scanning microscope).

Interaction of pAACs and arginine peptides with CXCR4 coreceptor: Interactions of pAACs and arginine-peptides with CXCR4 were determined by flow cytometry (FACS-Scan, Becton Dickinson) as previously described (Litovich (2001) supra). In brief, 0.5×10⁶ MT2 cells were washed with ice-cold PBS containing 0.1% sodium azide (wash buffer) and were incubated at 4° C. with anti-CXCR4 mAb, 12G5, conjugated to phycoerithrine (PE), in the absence or presence of different concentrations of pAACs and arginine peptides. After 30 minutes of incubation, the cells were washed with ice-cold wash buffer and fixed in PBS containing 1% paraformaldehyde. Non-specific fluorescence was assessed using an isotype control. For each sample 10,000 events were acquired. Data were analyzed and processed using CellQuest™ software (Becton Dickinson).

Selection of pAACs HIV-1 resistant isolates: MT2 cells (3×10⁵ cells in 1 ml) were preincubated with a selected pAACs at the IC₅₀ concentration for 30 minutes and were then infected with HIV-1_{IIIb} (5×10⁵ TCID₅₀). Culture fluids were replaced twice weekly with fresh medium containing the appropriate pAACs concentration. During the propagation of the virus, when at each cycle (at certain pAACs concentration) about 70% syncytium appeared, 250 μL of undiluted clarified culture supernatant, obtained from the HIV-infected cells, were added to 3×10⁵ fresh MT² cells in 1 ml final volume, containing two times higher pAACs concentration. From the final cycle of each experiment, the resistant virus was propagated as described above.

The EC₅₀ values of the pAACs against the resistant isolates were examined and compared to the wild type virus. After 24-26 cycles of selection of pAACs HIV-1-resistant isolates, genomic DNAs were purified from the infected MT2 cells.

A fragment of 648 bp of proviral HIV-1 DNA corresponding to the HIV-1 gp120 sequence was amplified by PCR with Taq DNA polymerase (Sigma, Rehovot, Israel) and the following forward and reverse primers, respectively: 5′-CACTTCTCCAATTGTCCCTCA-3′ and 5′-TGT TAAATGGCAGCCTAGCA-3′ (Biological Services, Weizmann Institute of Science). Amplified products were purified by gel electrophoresis on 2% agarose gel. Sequencing was carried out by using the forward primer with an ABI Prism, 3700 DNA analyzer, PE, Applied Biosystems, Hitachi.

Binding to Recombinant gp41: Enzyme-linked Immunosorbent assay (ELISA) was used in order to determine the capacity of the pAACs to bind to HIV-1 envelope glycoprotein 41 (gp41). 96 well microliter plates were coated overnight at 4° C. with recombinant gp41 antigen (a.a 466 to a.a 753 of the HIV-1 gp160 precursor, ViroGen Corporation, MA, USA; 1μg/ml). After washing with a buffer (PBS with 0.5% Tween-20, pH 7.4) four times, the plates were blocked by adding 200 μl per well of Assay diluent (PharMingen, San Diego, Calif., USA) for 1 hour at room temperature and re-washed with the buffer solution. 100 μl of 1:4 to 1:80,000 dilutions of sera samples obtained from HIV-1 sero-positive or sero-negative individuals, with and without 10 μM pAACs, were added to the wells. After 1 hour of incubation at room temperature, the plates were washed 4 times with a wash buffer and incubated for 1 hour at room temperature with Horseradish peroxidase (HRP) conjugated to goat anti-human IgG (Jackson Research Laboratories, Maine, USA; 1:10,000 dilution). After washing 4 times with a wash buffer, 100 μl of tetramethylbenzidine (TMB; PharMingen, San Diego, Calif., USA) was added in the dark and after 15 minutes of incubation at room temperature the reaction was stopped with 1M H₂SO₄. The absorbency at 450 nm was read after stopping the reaction. No hemolysis was noted up to concentrations of 100 μM for all the pAACs (data not shown)

Cellular uptake competition of pAA Cs with polyarginine-FITC: Phosphate-buffer saline (PBS) containing 0.5 μM FITC-labeled D-Arg-9-mer (Compound 12) with or without 10-, 40-, or 100-fold higher concentrations of non-labeled pAACs, are added to MT2 cells. After 5 or 15 minutes of incubation, the cells are washed and fixed in PBS containing 1% paraformaldehyde. The fluorescence is analyzed by flow cytometry.

Viral binding assay: HIV-1 viral particles are radiolabeled by endogenous reverse transcriptase (ERT), as previously described. The viral particles are incubated for 2 hours at 37° C. with MT2 or U937 cells in the presence of various concentrations of pAACs. Following the incubation and re-suspension of the cells in PBS, radioactivity is measured.

Competition between pAACs with the CXCR4 natural ligand SDF-1α and HIV-1 gp120: FITC-labeled pAACs are incubated at 4° C. with PM1, MT2 or PBMC cells with or without various concentrations of SDF-1α or with 5 μM recombinant HIV-1_{IIIB} gp102. After 30 minutes of incubation, the cells are washed with a wash buffer and analyzed by flow cytometry.

Competition between pAACs with the CCR5 chemokine RANTES: Competition between pAACs and RANTES are determined in cMAGI cells using flow cytometry according to a known procedure.

Antibacterial Activity Studies:

Minimal Inhibition Concentration (MIC) measurements: The Minimal Inhibitory Concentration (MIC) was generally determined by the Broth Microdilution method (based on the protocol described in Clinical and Laboratory Standard Institute (CLSI) NCCLS (2003) M7-A6), as follows:

A stock solution of each of the tested compounds was prepared by diluting the compound to a 2,560 μg/ml concentration with an appropriate solvent. The stock solution was then serially 2-fold diluted in 96-well microdilution trays using Cation-Adjusted Mueller-Hinton Broth (CAMHB) until reaching the desired range of concentrations, whereas each well contained double the final working concentration. Each row further had one growth control well (medium+inoculum only), and one sterility control well (medium+tested compound). Plates were stored at −70° C. until used.

The tested microorganisms were used as fresh overnight cultures on blood agar (for gram-positive organisms) or MacConkey agar (for gram-negatives). A standardized inoculum suspension was prepared by the direct colony suspension method, using sterile saline (equivalent to a 0.5 McFarland standard). The suspension was 100-fold and then 2-fold diluted, such that the addition of 50 μof the suspension to each of the tested wells resulted in a final inoculum concentration of approximately 5×10⁵ organisms per well.

Thus, 50 μl of the inoculum suspension were added to each tested well (except for the sterility control well), and inoculation was performed during 18-24 hours (depending on the tested microorganism or compound) at 35° C.

The MIC was determined by light transmittance using the Microtiter mirror-viewer with oblique lighting, as the highest dilution concentration of the tested compound which completely inhibited visible microorganism growth and is given both in mg/liter and in μM.

Using the above method, the antimicrobial activity of Neomycin B, L-Neo-9-mer (Compound 8), D-Neo-9-mer (Compound 14), D-Nea-9-mer (Compound 13), P55, NeoR6, D-Arg-9-mer (Compound 12) and L-Arg-9-mer (Compound 5) was tested.

The MIC values of gentamycin were determined by the Disk Diffusion test (also know as the “Kirby Bauer” or “Disk test”), according to the protocol described in “Performance Standards for Antimicrobial Susceptibility Testing; Fifteenth Informational Supplement (Volume 15 Number 1, Wilker et al., Eds. Clinical and Laboratory Standards Institute). In this method a paper disk soaked with 10 μM of gentamycin was placed over an inoculated plate. The gentamycin in each disc diffuses outward from the disc, thus diminishing its concentration as a function of its distance from the disc center. After incubation, the diameter of the zone of growth inhibition was measured and scored as either “sensitive” (or “susceptible” in terms of the susceptibility of the tested microorganism to such a treatment) for a diameter ≧15 millimeters, which is equivalent to a MIC≦4 mg/liter, or resistant (namely, the tested microorganism is resistant to the treatment) for a diameter ≦12 millimeters, which is equivalent to a MIC≧8 mg/liter), as described in Wilker et al. (supra, see, Tables 2A, 2B and 2C).

### EXPERIMENTAL RESULTS

Antiviral Activity:

The new pAACs were tested as anti-HIV agents. The effect of L, D and L/D peptides of different lengths (6- and 9-mers) and their aminoglycoside conjugates on several strains of HIV-1, including wild type, drug resistance and clinical isolates was examined.

Antiviral activity of the pAACs Neam-R6-55 (N55) and Paromo-R6-55 (P55): The hexa-arginine peptide conjugates of neamine and paromomycin, N55 and P55, respectively, which include the additional 55 Daltons organic moiety, were first studied. Anti-HIV activities of these two compounds were tested for a variety of M- and T-tropic HIV-1 isolates, including laboratory adapted T-tropic isolates, clinical isolates, as well as resistant HIV-1 strains. Table 2 below presents the antiviral activity (as the 50% effective concentration (EC₅₀ values)) of these exemplary pAACs. The data are the average of three independent experiments carried out in triplicate; ^{T}denotes T-tropic and ^{M} denotes M-tropic.

As shown in Table 2, the pAACs Neam-R6-55 (N55) and Paromo-R6-55 (P55) were found highly active in inhibiting a variety of HIV-I isolates, including laboratory adapted T- and M-tropic strains and clinical isolates. Importantly, these conjugates were found highly active also in inhibiting resistant strains, including NeoR6 resistant isolate (NeoR6r^{res}), with no significant differences between the neamine and paromomycin conjugates in the concentrations that caused a 50 % inhibition of viral production (EC₅₀ of 1-3 μM for compound N55 and 0.7-6 μM for compound P55).

TABLE 2

| | | | EC₅₀(μM) | |
|---|---|---|---|---|
| Viral Isolates | | | N55 | P55 |
| HIV-1 Laboratory | Wild Type | IIIB^{T} | 1.10 ± 0.25 | 0.75 ± 0.60 |
| Strains | | 2D^{T} | — | 1.25 ± 0.75 |
| | | LAI^{T} | 2.45 ± 1.30 | 6.65 ± 1.85 |
| | | Ba-L^{M} | 1.60 ± 0.5 | 2.15 ± 1.45 |
| | | SF162^{M} | 3.60 ± 1.2 | 3.00 ± 1.10 |
| | Resistant | NNRTI^{T} | 3.00 ± 1.50 | 0.80 ± 0.60 |
| | Virus | AZT^{T} | 3.10 ± 2.65 | 1.60 ± 0.40 |
| | | Protease^{T} | 1.60 ± 1.35 | 1.40 ± 0.15 |
| | | NeoR6^{T} | 3.10 ± 0.90 | 2.30 ± 1.05 |

| | | | |
|---|---|---|---|
| HIV-1 Clinical | Clade A^{T} | 1.80 ± 1.20 | 3.25 ± 0.55 |
| Isolates | Clade B^{T} | 3.60 ± 1.0 | 1.25 ± 1.15 |
| | Clade C^{T} | 2.70 ± 0.70 | 1.15 ± 0.85 |

It is noteworthy that N55 and P55 were also active against M-tropic strains Ba-L and SF162 (EC₅₀ of 1.6-3.6 μM) and against NeoR6 resistant virus (Litovchick et al. (2001) supra) with the EC₅₀ being approximately 3 μM for both compounds, suggesting that the polyarginine-aminoglycoside conjugates may obstruct HIV-1 replication by a different mechanism than NeoR6.

A small difference in the inhibition of HIV-1_{IIIB} by P55 and N55 (EC₅₀ of 0.7 and 1.1, respectively) was noticed in comparison to NeoR6^{res} (EC₅₀ of 2.3 and 3.1 μM, respectively). This is in contrast to the inhibition of NeoR6^{res} virus by NeoR6, which is approximately 46 times more resistant than HIV-1_{IIIB} virus to NeoR6 (Borkow et al. (2003a) supra). The sensitivity of these two new compounds to the other strains is similar to that of the NeoR6^{res} virus.

Anti-HIV activity of pAACs comprising L-arginine peptides (6- and 9-mers): Non-protected pAACs comprising the aminoglycosides neamine, paromomycin and neomycin, conjugated to 6- or 9-mer arginine (Set A in Table 1 above) were tested for their anti-HIV activity. Table 3 below presents the antiviral activity (as the 50% effective concentration (EC₅₀ values)) and the cytotoxicity of several L-peptides and their neamine, paromomycin and neomycin conjugates, as well as of an L/D-Arginine-neamine conjugate, against HIV-1_{IIIB} virus.

TABLE 3

| Compound (No.) | EC₅₀ (μM) | Cytotoxicity (μM) |
|---|---|---|
| L-Arg-6-mer (1) | 110 ± 20 | — |
| L-Arg-6-mer-neamine (2) | 70 ± 10 | 210 |
| L-Arg-6-mer-paromomycin (3) | 31 ± 10 | 160 |
| L-Arg-6-mer-neomycin (4) | 40 ± 12 | 200 |
| L-Arg-9-mer (5) | 33 ± 3 | 120 |
| L-Arg-9-mer-neamine (6) | 37.5 ± 2.5 | 175 |
| L-Arg-9-mer-paromomycin (7) | 31 ± 9 | 140 |
| L-Arg-9-mer-neomycin (8) | 30 ± 7 | 150 |
| L/D-9-mer-neamine (16) | 28 ± 2 | 160 |
| Neomycin | >200 | >300 |

As shown in Table 3, EC₅₀ values in the range of 30-70 μM were observed for the conjugates containing L-Arg-mers, whereas the EC₅₀ values of the respective L-arginine peptides (compounds 1 and 5) were 110 and 33 μM, respectively, and that of the free aminoglycoside neomycin was above 200 μM. The EC₅₀ of the mixed D/L nona-arginine neamine conjugate (compound 16) revealed a lower EC₅₀ as compared with L-arginine conjugates of the same chain length and the same core.

Thus, the non-conjugated aminoglycoside antibiotics neamine and paromomycin (data not shown) and the hexa-arginine peptide exhibited no inhibition activity in up to 50 μM concentrations, whereas the pAACs mostly displayed inhibition at concentrations of 40 μM and lower. Furthermore, the pAACs were found to be nontoxic, with the hexa-arginine-neomycin being non-toxic even at concentrations as high as 200 μM.

For comparison, the anti-viral activity of the pAAC that comprises a diarginine, L-Arg-2-mer-neamine (Compound 21) was similarly tested. An EC₅₀ value of about 46 μM was obtained, indicating a lower activity of this conjugate as compared with the corresponding nona-arginine conjugate of neamine. Anti-HIV activity of pAACs comprising D-arginine peptides (6- and 9-mers): Next, the antiviral activity of the parallel set of N-terminal acetylated D-arginine peptides (6- and 9-mers) and their conjugates with neamine and neomycin, (see, Set B in Table 1) was tested. Table 4 below presents the antiviral activity (as the 50% effective concentration (EC₅₀ values)) and the cytotoxicity of exemplary D-peptides and their neamnne and neomycin conjugates against HIV-1 clinical isolates and laboratory strains. The data are the average of three independent experiments carried out in triplicate. Cytotoxicity was measured by tryphan blue exclusion assay for MT2 cells.

TABLE 4

| | EC₅₀ (μM) | | | | | | | Cytotoxicity |
|---|---|---|---|---|---|---|---|---|
| Compound No. | IIIB | NeoR6^{Res} | Ba-L | Clade A | AZT^{Res} | Clade C | Protease^{Res} | (μM) |
| D-Arg-6-mer | 5.8 ± 1.7 | 6.8 ± 0.7 | >50 | 8.0 ± 1.0 | 15.2 ± 6.2 | 5.3 ± 0.3 | 4.9 ± 1.0 | 170 |
| (9) | | | | | | | | |
| D-Arg-6-mer- | 1.8 ± 0.1 | 2.6 ± 1.2 | >50 | 3.2 ± 1.3 | 7.0 ± 4.0 | 1.7 ± 0.1 | 2.2 ± 0.4 | 150 |
| neamine (10) | | | | | | | | |
| D-Arg-6-mer- | 2.1 ± 0.6 | 5.3 ± 2.5 | >50 | 5.7 ± 2.8 | 10.4 ± 4.1 | 3.0 ± 0.8 | 4.0 ± 1.0 | 155 |
| neomycin (11) | | | | | | | | |
| D-Arg-9-mer | 1.5 ± 0.6 | 2.0 ± 1.2 | >50 | 2.2 ± 0.3 | 9.7 ± 5.9 | 1.6 ± 0.4 | 1.4 ± 0.6 | 150 |
| (12) | | | | | | | | |
| D-Arg-9-mer- | 2.3 ± 0.5 | 3.0 ± 1.0 | >50 | 5.1 ± 2.9 | 10.4 ± 2.7 | 2.7 ± 0.5 | 2.4 ± 0.4 | 150 |
| neamine (13) | | | | | | | | |
| D-Arg-9-mer- | 1.3 ± 0.4 | 2.0 ± 0.5 | >50 | 2.1 ± 0.4 | 6.6 ± 5.9 | 1.2 ± 0.5 | 1.5 ± 0.4 | 120 |
| neomycin (14) | | | | | | | | |

As shown in Table 4, the D-arginine-containing peptides and conjugates all inhibited a variety of HIV-1 isolates including T-tropic laboratory adapted and clinical isolates as well as resistant strains, including NeoR6^{res} virus, with EC₅₀ values in the range of 1.2-15.2 μM. No significant differences were observed for the antiviral potency of pAACs containing 6- or 9-mers D-arginine, or between a core of neamine or neomycin. There were also no significant differences between the capacities of the D-compounds to inhibit HIV-1_{III B} wild type (wt) virus compared to the NeoR6^{res} variant. While the nona-D-arginine-N-acetate revealed similar antiviral activity as its conjugates with aminoglycosides, the 6-mer-D-arginine-N-acetate antiviral activity was somewhat lower than that of the 9-mer-N-acetate and of the aminoglycoside 6-mer and 9-mer aminoglycoside conjugates. For example, the EC₅₀ of D-Arg-6-mer with HIV-1_{IIIB} was 5.8±1.7 μM and that of D-Arg-6-mer-neamine was 1.8±0.1 μM. A similar ratio was obtained with the other viruses tested.

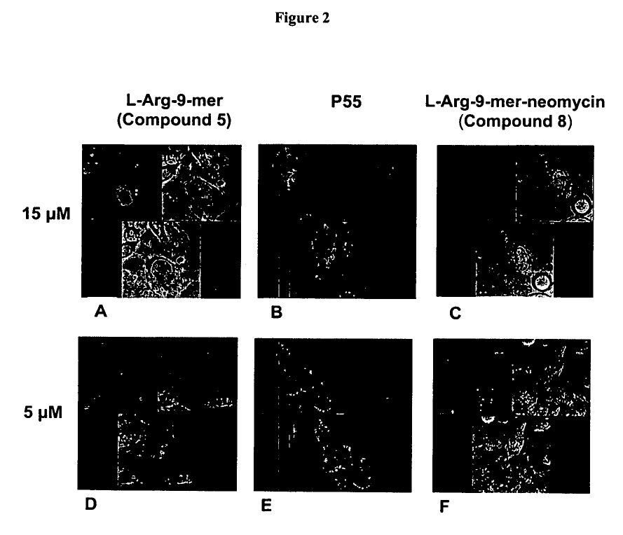

Cellular uptake of pAA Cs: The cellular uptake and distribution of fluorescently labeled (FITC derivatives) novel pAACs, were studied, using confocal microscopy. Exemplary images obtained in these studies, of live MT2 cells, incubated for 30 minutes at 37° C. with the fluorescent derivatives (FITC) of P55, L-oligoarginine-9-mer (Compound 5) and its neomycin conjugate (Compound 8) at 15 μM and at 5 μM are presented in FIGS. 2A-C and 2D-F, respectively.

As shown in FIGS. 2B and 2E, P55 readily penetrated the cells (even at 5 μM) and accumulated intracellularly and in the nucleus. Similarly, the FITC derivative of N-terminal acetate D-arginine and the neamine and neomycin conjugates thereof displayed efficient cell uptake even in low concentration (data not shown). However, the L-Arg-9-mer displayed lower uptake efficiency (FIGS. 2C and 2F). It is noteworthy that increasing the concentration of the FITC-L-peptides aminoglycoside conjugates to 30 μM (data not shown) did result in a cellular uptake and internalization under this short time of incubation. A similar low uptake efficiency was found, as expected, for the respective L-arginine peptide (FIGS. 2A and 2D).

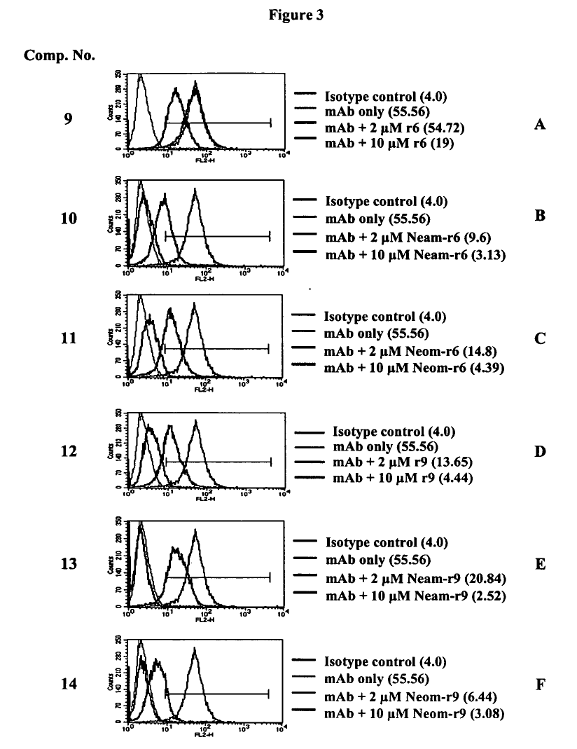

Inhibition of anti-CXCR4 mab binding to cells by pAACs: The ability of the novel pAACs presented herein to interact with CXCR4 was studied. The capacity of the various pAACs to block the binding of the PE labeled 12G5 mAb to CXCR4 in MT2 cells was examined and the results are presented in Table 5 below and in FIG. 3. As shown, for example, in FIG. 3, in the presence of D-Arg-6-mer-neamine (Compound 10), the median fluorescent intensity (MFI) of 12G5 mAb binding to MT2 cells was 55.56, while of the isotype control was 4.0. In the presence of 2 and 10 μM of Compound 10, the MFI of the mAb binding to cells was reduced to 9.6 and 3.3 respectively, thus achieving 91% and 100% inhibition, respectively.

Table 5 below presents the percent of inhibition of 12G5 mAb binding to CXCR4 by several exemplary pAACs, categorized as containing non-protected L-peptides; containing N-protected D-peptides; and including the N55 and P55 conjugates.

TABLE 5

| | |
|---|---|
| Compound (No.) | % Inhibition |

| | | |
|---|---|---|
| | 20 μM | 80 μM |
| L-Arg-6-mer-neamine (2) | 35.4 | — |
| L-Arg-9-mer (5) | — | 52.8 |
| L-Arg-9-mer-neamine (6) | — | 30.1 |
| | 2 μM | 10 μM |
| D-Arg-6-mer (9) | 1.6 | 70.9 |
| D-Arg-6-mer-neamine (10) | 91.1 | 100 |
| D-Arg-6-mer-neomycin (11) | 79 | 99.3 |
| D-Arg-9-mer (12) | 81.3 | 99.6 |
| D-Arg-9-mer-neamine (13) | 67.3 | 100 |
| D-Arg-9-mer-neomycin (14) | 95.3 | 100 |
| | 1 μM | 5 μM |
| N55 | 92.8 | 100 |
| P55 | 70.8 | 100 |

As shown in Table 5, the D-arginine-aminoglycoside conjugates exhibited an inhibitory activity higher by at least 30-fold stronger as compared with the L-peptide conjugates. The N55 and P55 exerted similar inhibition of anti-CXCR4 mAb binding to cells as the D-arginine aminoglycoside conjugates.

The inhibition of binding of mAb 12G5 to CXCR4 by each one of the novel pAACs, as well as by P55 and N55, supports the notion that the polyarginine-aminoglycoside conjugates interact with CXCR4, and that the D-polyarginines (both 6- and 9-mers)-aminoglycoside conjugates were more potent compared to the L-polyarginines. No significant difference in inhibition of the CXCR4 mAB binding was found between the 6- and 9-mers of D-arginine-aminoglycosides conjugates or between these peptides conjugated to neamine or to neomycin. The only significant difference found in this group, was between the peptide D-6-mer and D-9-mer N-terminal acetate. This is in contrast to the aminoglycoside conjugates thereof, where no significant changes were observed.

Comparing the results to those obtained with NeoR6 (see, Litovchick 2001 supra) show that while 2.5 μM (NeoR6) caused 66% inhibition of mAb 12G5 binding to CXCR4, 2 μM of r6-D-arginine-neomycin caused 91.1% inhibition of mAb 12G5 binding to CXCR4, and r9-neomycin caused a 95.3% inhibition.

Interaction of pAACs with gp41:

The HIV-1 gp41 envelope sub-unit is critical for HIV-1 binding and fusion with target cells (Borkow and Lapidot (2005) supra). Previously, mutations in the gp41 (at positions 668 and 672 of the gp160 precursor) of NeoR6^{res} virus were found, indicating that AACs may exert their antiviral activity by interacting with this viral glycoprotein (Borkow et al. (2003a and 2003b) supra).

Thus, the capacity of the novel pAACs presented herein to interact with recombinant gp41 antigen (a.a 466 to a.a 753 of the HIV-1 gp160 precursor) was now examined by ELISA as described in the Methods section hereinabove. No interaction of the pAACs with gp41 was detected.

Hemolysis Studies:

In order to study the possibility of intra-venal administration of pAACs, the hemolytic activity of the pAACs was studied as described in the Methods section. No hemolysis was noted up to concentrations of 100 μM for all the pAACs (data not shown).

Antibacterial Activity:

The antibacterial activity of exemplary pAACs according to the present embodiments: L-Neo-9-mer (Compound 8), D-neo-9-mer (Compound 14), D-nea-9-mer (Compound 13) and P55, was tested and compared to that of a non-polymeric arginine-aminoglycoside conjugate (NeoR6, see above), to free neomycin B, togentamycin and to the non-conjugated D-Arg-9-mer and L-Ard-9-mer peptides (Compounds 12 and 5, respectively). Gram-positive and Gram-negative bacteria, reference strains and clinical isolates, were treated with increasing concentrations of the tested compounds and the Minimum Inhibitory Concentration (MIC) values were determined as described hereinabove.

Table 6 below presents the results obtained for the antimicrobial activity of L-Neo-9-mer (Compound 8) and L-Par-6-mer-55 (P55), as compared to the activity of NeoR6 and neomycin B. ^{s} denotes a microorganism sensitive to either gentamycin (noted as gentamycin^{s}), meropenem (noted as meropenem^{s}) or amikacin (noted as amikacyn^{s}). ^{r} denotes a microorganism resistant to either gentamycin (noted as gentamycin^{r}), meropenem (noted as meropenem^{r}) or amikacin (noted as amikacyn^{r}). MIC values are given both in mg/l and in μM (in parenthesis) and are a mean of two individual experiments carried out in triplicates.

TABLE 6

| | Minimum Inhibitory Concentration | | | |
|---|---|---|---|---|
| | mg/liter (μM) | | | |
| | | | L-Par-6- | |
| | | L-Neo-9-mer | mer 55 | |
| Organisms | NeoR6 | (Compound 8) | (P55) | Neomycin |
| Gram Positive Bacteria | | | | |
| Reference Strains | | | | |
| Staphylococcus aureus ATCC | 2.0 (0.88) | — | — | 1.5 (1.65) |
| 24213 | | | | |
| Staphylococcus aureus ATCC | 6.2 (2.73) | — | — | — |
| 6538 | | | | |
| Staphylococcus epidermidis | 0.8 ((0.35) | — | — | — |
| ATCC12228 | | | | |
| Enterococcus faecalis ATCC | >128.0 (>56.0) | — | — | 128.0 (140.81) |
| 29212 | | | | |
| Streptococcus pyogenes | 64.0 (28.00) | — | — | — |
| ATCC 19615 | | | | |
| Streptococcus bovis ATCC | 64.0 (28.0) | | — | — |
| 9809 | | | | |
| Bacillus subtilis ATCC 6633 | 1.5 (0.66) | — | — | — |
| Clinical Isolates | | | | |
| S. aureus MSSA | 2.0 (0.88) | 4.0 (1.37) | — | 1.0 (1.10) |
| S. aureus MRSA | 2.0 (0.88) | 2.0 (0.68) | — | 1.0 (1.10) |
| S. epidermidis MSSE | 1.5 (0.66) | 0.5 (0.17) | 0.5 (0.23) | 0.5 (0.55) |
| S. epidermidis MRSE | 0.7 (0.31) | 0.5 (0.17) | 0.5 (0.23) | 0.5 (0.55) |
| Strep. Pneumoniae | >128.0 (>56.0) | — | — | — |
| Strep. Pyogenes (gr. A) | 64.0 (28.0) | — | — | 16.0 (17.60) |
| Strep. Agalactiae (gr. B) | >128.0 (>56.0) | — | — | — |
| Enterococcus faecalis | ≧128.0 (≧56.0) | 256.0 (87.67) | — | 128.0 (140.81) |
| Enterococcus faecium | >128.0 (>56.0) | 32.0 (10.96) | — | 256.0 (281.63) |
| Listeria monocytogenes | 2.0 (0.88) | — | — | — |
| S. epidermidis B32954 | — | 0.5 (0.17) | — | 0.5 (0.55) |
| Enterococcus faecalis | — | 32.0 (10.96) | — | 64.0 (70.41) |
| B84939-vancomycin^{r} | | | | |

Gram Negative Bacteria

| | | | | |
|---|---|---|---|---|
| Reference Strains | | | | |
| Escherichia coli ATCC 25922 | 2.0 (0.88) | — | — | 1.0 (1.10) |
| Pseudomonas aeruginosa | 128.0 (56.00) | — | — | 128.0 (140.81) |
| ATCC 27853 | | | | |
| Klebsiella pneumoniae ATCC | 2.0 (0.88) | — | — | 0.7 (0.77) |
| 13883 | | | | |
| Proteus mirabilis ATCC 7002 | 2.0 (0.88) | — | — | 1.5 (1.65) |
| Acinetobacter baumannii | 16.0 (7.05) | | — | 6.0 (6.60) |
| ATCC 19606 | | | | |
| Moraxella catarrhalis ATCC | 0.37 (0.16) | — | — | 0.25 (0.28) |
| 25238 (Branhamella) | | | | |
| Clinical Isolates | | | | |
| E. Coli-ESBL negative | 2.0 (0.88) | 4.0 (1.37) | 4.0 (1.81) | 1.0 (1.10) |
| E. Coli-ESBL positive | 4.0 (1.76) | 8.0 (2.74) | — | 4.0 (4.40) |
| K. pneumoniae-ESBL | 2.0 (0.88) | 3.0 (1.03) | 4.0 (1.81) | 0.7 (0.77) |
| negative | | | | |
| K. pneumoniae-ESBL | 8.0 (3.52) | 1.5 (0.51) | — | 1.5 (1.65) |
| positive | | | | |
| Serratia marcescens | 4.0 (1.76) | 4.0 (1.37) | — | 1.0 (1.10) |
| Enterobacter cloacae | 8.0 (3.52) | 8.0 (2.74) | — | 4.0 (4.40) |
| Enterobacter aerogenes | 8.0 (3.52) | 8.0 (2.74) | — | 0.7 (0.77) |
| P. aeruginosa-gentamicin^{s}, | 32.0 (14.08) | 48.0 (16.44) | — | 16.0 (17.60) |
| meropenem^{s} | | | | |
| P. aeruginosa-gentamicin^{r}, | 128.0 (56.00) | 128.0 (43.83) | — | 128.0 (140.81) |
| meropenem^{r} | | | | |
| A. baumannii-amikacin^{s}, | 4.0 (1.76) | 6.0 (2.06) | — | 3.0 (3.30) |
| meropenem^{s} | | | | |
| A. baumannii-amikacin^{r}, | 128.0 (56.00) | 128.0 (43.83) | — | 96.0 (105.61) |
| meropenem^{r} | | | | |
| E. coli U23773^{r} | — | 96.0 (32.88) | 128 (57.92) | 128.0 (140.81) |
| Enterobacter cloacae W25161 | — | 8.0 (2.74) | — | 3.0 (3.30) |
| P. aeruginosa R17093 | — | 128.0 (43.83) | — | 192.0 (211.22) |
| K. pneumoniae B2511 | — | — | — | 1.5 (1.65) |
| A. baumannii R16998 | | 128.0 (43.83) | | 32.0 (35.20) |

As is shown in Table 6, no significant differences were observed in the antibacterial activity of L-Neo-9-mer and L-Par-6-mer-55. More importantly, these two tested pAACs exhibited in most cases a better antibacterial activity as compared with neomycin and NeoR6. For example, for P. aeruginosa R17093, the MIC of L-Neo-9-mer was 43.83 μM whereby that of neomycin was 211.22 μM. In another example, for E. coli U23773^{r}, the MIC of L-Neo-9-mer and of L-Par-6-mer-55 were 32.88 and 57.92 μM, respectively, whereby that of neomycin was 140.81 μM. In yet another example, for A. baumannii-amikacin^{r}, meropenem^{r}, the MIC of L-Neo-9-mer was 57.92 μM, whereby that of neomycin was 105.61 μM.

Table 7 below presents the results obtained for the antimicrobial activity of D-Nea-9-mer (Compound 13), D-Neo-9-mer (Compound 14), D-Arg-9-mer and L-Arg-9-mer (Compound 5), compared with that of gentamycin (measured by the disk method described hereinabove at a concentration of 10 μM). MIC values are given both in mg/l and in μM (in parenthesis) and are a mean of two individual experiments carried out in triplicates. ^{r} denotes a microorganism resistant to either gentamycin (noted as gentamycin^{r}), meropenem (noted as meropenem^{r}) or amikaycin (noted as amikacyn^{r}). In the gentamycin column, ^{r} denotes resistance of the tested strain (disk method diameter ≦12 millimeters, equivalent to a MIC ≧8 mg/liter) and s denotes sensitivity of the tested strain (disk method diameter ≧15 millimeters, equivalent to a MIC ≦4 mg/liter).

TABLE 7

| | Minimum Inhibitory Concentration | | | | |
|---|---|---|---|---|---|
| | mg/liter (μM) | | | | |
| | | D-Arg-9-mer- | D-Arg-9-mer- | | |
| | | neamine | neomycin | | |
| | Gentamycin | (Compound | (Compound | D-Arg-9-mer | L-Arg-9-mer |
| Organisms | (disk) | 13) | 14) | (Compound 12) | (Compound 5) |
| Gram | | | | | |
| Positive | | | | | |
| Bacteria | | | | | |
| Reference | | | | | |
| Strains | | | | | |
| Staphylococcus | | 2.0 (0.80) | 32.0 (11.02) | 128.0 (63.82) | 128.0 (63.25) |
| aureus | | | | | |
| ATCC 24213 | | | | | |
| Enterococcus | | | 128.0 (44.11) | 64.0 (31.91) | 96.0 (31.62) |
| faecalis ATCC | | | | | |
| 29212 | | | | | |
| Streptococcus | | | 24.0 (8.27) | 128.0 (63.82) | 128.0 (63.25) |
| bovis ATCC | | | | | |
| 9809 | | | | | |
| Clinical | | | | | |
| Isolates | | | | | |
| S. aureus | r | 1.5 (0.60) | 16.0 (5.51) | >128.0 (63.82) | 128.0 (63.25) |
| MSSA | | | | | |
| S. aureus | s | 1.5 (0.60) | 32.0 (11.02) | >128.0 (63.82) | >128.0 (63.25) |
| MRSA | | | | | |
| S. epidermidis | s | 0.5 (0.20) | 1.5.0 (0.52) | 8.0 (3.99) | 12.0 (5.93) |
| MSSE | | | | | |
| S. epidermidis | s | 0.5 (0.20) | 8.0 (2.76) | 96.0 (47.86) | 32.0 (15.81) |
| MRSE | | | | | |
| Enterococcus | | | 64.0 (22.05) | 128.0 (63.82) | 128.0 (63.25) |
| faecalis | | | | | |
| Enterococcus | | | 24.0 (8.27) | | |
| faecium | | | | | |
| Gram | | | | | |
| Negative | | | | | |
| Bacteria | | | | | |
| Reference | | | | | |
| Strains | | | | | |
| Escherichia | | 6.0 (2.40) | 32.0 (11.02) | 128.0 (63.82) | >256.0 (126.50) |
| coli ATCC | | | | | |
| 25922 | | | | | |
| Pseudomonas | | 96.0 (38.55) | >256.0 (88.22) | >256.0 (127.64) | >256.0 (126.50) |
| aeruginosa | | | | | |
| ATCC 27853 | | | | | |
| Klebsiella | | 1.5 (0.60) | 12.0 (4.14) | 96.0 (47.86) | >256.0 (126.50) |
| pneumoniae | | | | | |
| ATCC 13883 | | | | | |
| Proteus | | 8.0 (3.20) | 64.0 (22.05) | >256.0 (127.64) | >256.0 (126.50) |
| mirabilis | | | | | |
| ATCC 7002 | | | | | |
| Acinetobacter | | 16.0 (6.40) | >256.0 (88.22) | >256.0 (127.64) | >256.0 (126.50) |
| baumannii | | | | | |
| ATCC 19606 | | | | | |
| Moraxella | | | 8.0 (2.76) | 16.0 (7.98) | 32.0 (15.81) |
| catarrhalis | | | | | |
| ATCC 25238 | | | | | |
| (Branhamella) | | | | | |
| Clinical | | | | | |
| Isolates | | | | | |
| E. Coli-ESBL | s | 6.0 (2.40) | 16.0 (5.51) | 128.0 (63.82) | >256.0 (126.50) |
| negative | | | | | |
| E. Coli-ESBL | r | 8.0 (3.20) | 24.0 (8.27) | 64.0 (31.91) | 256.0 (126.50) |
| positive | | | | | |
| K. pneumoniae- | s | 3.0 (1.20) | 64.0 (22.05) | 256.0 (127.64) | >256.0 (126.50) |
| ESBL negative | | | | | |
| K. pneumoniae- | r | 3.0 (1.20) | 32.0 (11.02) | 48.0 (23.93) | >256.0 (126.50) |
| ESBL positive | | | | | |
| Enterobacter | | 8.0 (3.20) | 64.0 (22.05) | 128.0 (63.82) | >256.0 (126.50) |
| cloacae | | | | | |
| Enterobacter | r | 16 (6.40) | 32.0 (11.02) | 128.0 (63.82) | >256.0 (126.50) |
| aerogenes | | | | | |
| P. aeruginosa- | | 48.0 (19.28) | >256.0 (88.22) | >256.0 (127.64) | >256.0 (126.50) |
| gentamicin^{s}, | | | | | |
| meropenem^{s} | | | | | |
| P. aeruginosa- | | 96.0 (38.55) | >156.0 (53.76) | >256.0 (127.64) | >256.0 (126.50) |
| gentamicin^{r}, | | | | | |
| meropenem^{r} | | | | | |
| A. baumannii- | r | 12.0 (4.8) | 128.0 (44.11) | 256.0 (127.64) | >256.0 (126.50) |
| amikacin^{s}, | | | | | |
| meropenem^{s} | | | | | |
| A. baumannii- | r | 96.0 (38.55) | >256.0 (88.22) | >256.0 (127.64) | >256.0(126.50) |
| amikacin^{r}, | | | | | |
| meropenem^{r} | | | | | |
| Enterobacter | r | 24.0 (9.64) | | | |
| cloacae | | | | | |
| W25161^{r} | | | | | |
| A. baumannii | r | 128.0 (51.41) | | | |
| R16998^{r} | | | | | |
| Enterococcus | r | 64.0 (25.70) | | | |
| faecalis- | | | | | |
| vancomycin^{r} | | | | | |
| E. Coli | r | 6.0 (2.40) | | | |
| B3095141^{r} | | | | | |
| A. baumannii | | | 64.0 (22.05) | | |
| E11436 | | | | | |
| Serratia | | | 64.0 (22.05) | >256.0 (127.64) | >256.0 (126.50) |
| marcescens | | | | | |
| E. coli | | | 48.0 (16.54) | 96.0 (47.86) | >256.0 (126.50) |
| U3075921 | | | | | |
| E. coli U23773 | | | 32.0 (11.02) | 64.0 (31.91) | |
| VRE faecium | | | | >128.0 (63.82) | >128.0 (63.25) |

As is shown in Table 7, a considerable amount of microorganism strains that were found resistant to gentamycin, did not show such a resistance when treated with the tested pAAC (see for example compound 13, for K. pneumoniae-ESBL positive, A. baumannii-amikacin^{s}, meropenem^{s}, and E. Coli B3095141^{r}).

As is also shown in Table 7, the MIC values of the D-arginine-containing conjugates, D-Arg-9-mer-neamine and D-Arg-9-mer-neomycin, were in most cases lower than the MIC values of the corresponding D-Arg-9-mer-peptide, demonstrating the superior performance thereof. The improved performance (lower MIC values) of the neamine conjugates (see, for example, Compound 13) was the most significant. These results are in accordance with previous studies which demonstrated that the aminoglycoside core has an important role in the antiviral potency of AACs (Litovchick et al. (2000), supra).

As is further shown in Tables 6 and 7, no significant difference was observed in the antibacterial activity of D-arginine-containing conjugates and L-arginine-containing conjugates. See for example, the MIC values for both E. Coli-ESBL negative and E. Coli-ESBL positive. The MIC values presented in Tables 6 and 7, for all the tested L- and D-conjugates, respective to the tested bacterial strain, are summarized in Table 8 below.

TABLE 8

| | | |
|---|---|---|
| Reference Strains | Clinical Isolates | MIC (μM) |

Gram Positive Bacteria

| | | |
|---|---|---|
| Staphylococcus aureus ATCC 24213 | S. aureus MSSA | 0.16-3.52 |
| Staphylococcus epidermidis | S. aureus MRSA | |
| ATCC12228 | S. epidermidis MSSE | |
| Bacillus subtilis ATCC6633 | S. epidermidis MRSE | |
| | Listeria monocytogenes | |
| | S. epidermidis B32954 | |
| Staphylococcus aureus ATCC 6538 | Strep. Pyogenes (gr. A) | 6.6-28 |
| Streptococcus pyogenes ATCC19615 | Enterococcus faecium | |
| Streptococcus bovis ATCC9809 | Enterococcus faecalis B84939- | |
| | vancomycin^{r} | |
| Enterococcus faecalis ATCC 29212 | Strep. Pneumoniae | 32-56 |
| | Strep. Agalactiae (gr. B) | |
| | Enterococcus faecalis | |

Gram Negative Bacteria

| | | |
|---|---|---|
| Escherichia coli ATCC | E. Coli-ESBL negative | 0.16-3.52 |
| Klebsiella pneumoniae ATCC 13883 | E. Coli-ESBL positive | |
| Proteus mirabilis ATCC 7002 | K. pneumoniae-ESBL negative | |
| Moraxella catarrhalis ATCC 25238 | K. pneumoniae-ESBL positive | |
| (Branhamella) | Serratia marcescens | |
| | Enterobacter aerogenes | |
| | A. baumannii-amikacin^{s}, meropenem^{s} | |
| | k. pneumoniae B2511 | |
| | Enterobacter cloacae W25161 | |
| Acinetobacter baumannii ATCC 19606 | Enterobacter cloacae | 6.6-28 |
| | P. aeruginosa-gentamicin^{s}, meropenem^{s} | |
| Pseudomonas aeruginosa ATCC | P. aeruginosa-gentamicin^{r}, meropenem^{r} | 32-56 |
| | A. baumannii-amikacin^{r}, meropenem^{r} | |
| | E. coli U23773^{r} | |
| | P. aeruginosa R17093 | |
| | A. baumannii R16998 | |

As shown in Table 8, approximately 50% of the tested gram-positive strains and clinical isolates were sensitive to the various polyarginine-aminoglycoside conjugates with MIC values in the range of 0.6-3.52 μM, while the others were less sensitive with MIC usually in the range of 6.6-28 μM and a few in the range of 32-56 μM. The Gram-negative reference strains and clinical isolates revealed higher sensitivity to the arginine-aminoglycosides conjugates presented herein, and approximately two thirds thereof (including six clinical isolates) had MIC values in the range of 0.6-6.4 μM.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A conjugate comprising a first moiety and a second moiety being covalently linked therebetween, wherein said first moiety comprises at least one saccharide unit and said second moiety comprises at least two basic amino acid residues.

2. The conjugate of claim 1, wherein said first moiety is selected from the group consisting of a monosaccharide, an oligosaccharide and a polysaccharide.

3. The conjugate of claim 2, wherein said oligosaccharide is an aminoglycoside antibiotic.

4. The conjugate of claim 3, wherein said aminoglycoside antibiotic is selected from the group consisting of neomycin, kanamycin, sisomycin, fortimycin, paromomycin, neamine and gentamycin.

5. The conjugate of claim 3, wherein said second moiety is linked to an aminoalkyl group of said aminoglycoside.

6. The conjugate of claim 5, wherein said second moiety is linked to said aminoalkyl group via an amide bond.

7. The conjugate of claim 1, wherein said second moiety comprises at least six basic amino acid residues.

8. The conjugate of claim 7, wherein said second moiety comprises from 6 to 9 basic amino acid residues.

9. The conjugate of claim 1, wherein said second moiety is a peptide comprising said at least two basic amino acid residues.

10. The conjugate of claim 7, wherein said second moiety is a peptide comprising said at least six basic amino acid residues.

11. The conjugate of claim 8, wherein said second moiety is a peptide comprising from 6 to 9 basic amino acid residues.

12. The conjugate of claim 9, wherein said peptide consists essentially of said basic amino acid residues.

13. The conjugate of claim 1, wherein said basic amino acid residues are selected from the group consisting of arginines, lysines, histidines, omithines and any combinations thereof.

14. The conjugate of claim 1, wherein said basic amino acid residues are arginine residues.

15. The conjugate of claim 1, wherein said basic amino acid residues are selected from the group consisting of L-amino acid residues, D-amino acid residues and combinations thereof.

16. The conjugate of claim 1, wherein said basic amino acid residues are D-amino acid residues.

17. The conjugate of claim 1, wherein said basic amino acid residues are L-amino acid residues.

18. The conjugate of claim 14, wherein said basic amino acid residues are L-arginine residues.

19. The conjugate of claim 14, wherein said basic amino acid residues are D-arginine residues.

20. The conjugate of claim 18, further comprising at least one organic residue having a molecular weight of about 55 daltons.

21. A process of preparing the conjugate of claim 1, the process comprising:
coupling a first compound having at least one saccharide unit and a second compound having at least two basic amino acid residues, thereby obtaining the conjugate.

22. The process of claim 21, wherein said coupling is effected in the presence of a coupling agent.

23. The process of claim 22, wherein said coupling agent is a peptide coupling agent.

24. The process of claim 23, wherein said at least one saccharide unit comprises an aminoalkyl group and said coupling is effected via said aminoalkyl group, such that the process further comprises, prior to said coupling:
providing a compound having at least one saccharide unit and at least one aminoalkyl group attached to said saccharide unit, wherein any non-alkylamino groups in said compound are protected.

25. The process of claim 24, wherein providing said compound having at least one saccharide unit and at least one aminoalkyl group attached to said saccharide unit, wherein any non-alkylamino groups in said compound are protected comprises:
selectively protecting said at least one aminoalkyl group with a first protecting group;
selectively protecting said non-alkylamino groups with a second protecting group; and
selectively removing said first protecting group.

26. The process of claim 25, wherein said first protecting group is derived from a bulky protecting compound.

27. The process of claim 26, wherein said bulky protecting compound is selected from the group consisting of tritylhalide and N-(tert-butoxycarbonyloxy)-5-norbornene-endo-2,3 -dicarboximide.

28. The process of claim 21, wherein said compound having said basic amino acids comprises at least one third protecting group protecting at least one functional group in said compound and the process further comprising removing said third protecting group.

29. The process of claim 28, wherein removing said third protecting group is effected subsequent to said coupling.

30. A pharmaceutical composition comprising, as an active ingredient, the conjugate of claim 1 and pharmaceutically acceptable carrier.

31. The pharmaceutical composition of claim 30, being packaged in a packaging material and identified in print, in or on said packaging material, for use in the treatment of a medical condition associated with an infectious microorganism.

32. The pharmaceutical composition of claim 31, wherein said infectious microorganism is selected from the group consisting of a virus and a bacterial strain.

33. The pharmaceutical composition of claim 32, wherein said virus is HIV.

34. The pharmaceutical composition of claim 33, wherein said medical conditions is selected from the group consisting of AIDS and an AIDS manifestation.

35. The pharmaceutical composition of claim 31, further comprising at least one antiviral agent.

36. The pharmaceutical composition of claim 31, further comprising at least one antibacterial agent.

37. The pharmaceutical composition of claim 31, wherein said bacterial strain is a resistant bacterial strain.

38. The pharmaceutical composition of claim 37, wherein said bacterial strain is selected from the group consisting of a Gram positive strain and a Gram negative strain.

39. The pharmaceutical composition of claim 38, wherein said gram negative bacterial strain is selected from the group consisting of Escherichia coli, Pseudomonas aeruginosa, Klebsiella pneumoniae, Proteus mirabilis, Acinetobacter baumannii, Moraxella catarrhalis, Serratia marcescens, Enterobacter cloacae and Enterobacter aerogenes.

40. The pharmaceutical composition of claim 38, wherein said gram positive bacterial strain is selected from the group consisting of Staphylococcus aureus, Staphylococcus epidermidis, Streptococcus pyogenes, Streptococcus bovis, Streptococcus Pneumoniae, Streptococcus Pyogenes, Streptococcus Agalactiae, Bacillus subtilis, Enterococcus faecalis, Enterococcus faecium and Listeria monocytogenes.

41. A method of treating a medical condition associated with an infectious microorganism, the method comprising administering to a subject in need thereof a therapeutically effective amount of the conjugate of claim 1.

42. The method of claim 41, wherein said infectious microorganism is selected from the group consisting of a virus and a bacterial strain.

43. The method of claim 42, wherein said virus is HIV.

44. The method of claim 43, wherein said medical condition is selected from the group consisting of AIDS and an AIDS manifestation.

45. The method of claim 41, wherein said bacterial strain is a resistant bacterial strain.

46. The method of claim 45, wherein said bacterial strain is selected from the group consisting of a Gram positive strain and a Gram negative strain.

47. The method of claim 46, wherein said gram negative bacterial strain is selected from the group consisting of Escherichia coli, Pseudomonas aeruginosa, Klebsiella pneumoniae, Proteus mirabilis, Acinetobacter baumannii, Moraxella catarrhalis, Serratia marcescens, Enterobacter cloacae and Enterobacter aerogenes.

48. The method of claim 46, wherein said gram positive bacterial strain is selected from the group consisting of Staphylococcus aureus, Staphylococcus epidermidis, Streptococcus pyogenes, Streptococcus bovis, Streptococcus Pneumoniae, Streptococcus Pyogenes, Streptococcus Agalactiae, Bacillus subtilis, Enterococcus faecalis, Enterococcus faecium and Listeria monocytogenes.

49. The method of claim 41, further comprising administering to the subject at least one antiviral agent.

50. The method of claim 41, further comprising administering to the subject at least one antibacterial agent.
